(19) United States
(12) Patent Application Publication
(10) Pub. No.: US 2025/0366745 A1
(43) Pub. Date: Dec. 4, 2025

(54) PRESSURE PROBE DEVICE AND METHOD FOR ASSESSMENT OF VASCULAR HEALTH

(71) Applicant: NEW YORK UNIVERSITY, New York, NY (US)

(72) Inventors: Nisha Maheshwari, New York, NY (US); Alessandro Marone, Jersey City, NJ (US); Andreas Hielscher, Brooklyn, NY (US); Stephen HyunKeol Kim, Cresskill, NJ (US)

(21) Appl. No.: 19/220,980

(22) Filed: May 28, 2025

Related U.S. Application Data
(60) Provisional application No. 63/652,817, filed on May 29, 2024.

(51) Int. Cl. A61B 5/1455; A61B 5/024; A61B 5/1495

(52) U.S. Cl. CPC A61B 5/14552; A61B 5/02427; A61B 5/1495; A61B 2562/0247

(57) ABSTRACT

A medical pressure probe device comprises a body having a proximal end and a distal end, with a distal face, at least one optical sensor and at least one light source positioned on the distal face of the body, and at least one force sensor positioned inside the body, and a handle interfaced with the body and movable relative to the body along a path between a neutral position and an engaged position, the handle comprising a spring mechanism that contacts the at least one force sensor when the handle is in the engaged position to measure the applied pressure on the body from the handle, wherein the spring mechanism biases the handle to the neutral position.

### CROSS-REFERENCE TO RELATED APPLICATIONS

This application claims priority to U.S. Provisional Application No. 63/652,817 filed on May 29, 2024, incorporated herein by reference in its entirety.

### BACKGROUND OF THE INVENTION

Peripheral arterial disease (PAD) affects at least 8.5 million people in the United States alone [H. Johnston-Cox et al., Fuster and Hurst's The Heart, 15th ed., Chapter 26, V. Fuster, et al. Eds. McGraw Hill (2022)]. The disease is caused by a buildup of plaque in the arteries and predominantly affects the lower extremities. Early symptoms include discomfort or pain in the legs and feet. If left untreated, patients may experience more severe symptoms including gangrene, non-healing wounds, and amputation [A. Stoyioglou and M. R. Jaff, J. Vasc. Interv. Radiol. 15(11), 1197-1207 (2004); H. L. Gornik and J. A. Beckman, Circulation 111(13), e169-e172 (2005); R. A. G. Patel et al., Curr. Probl. Cardiol. 45(7), 100402-100429 (2020); J. D. Santilli and S. M. Santilli, Am. Fam. Physician. 59(7), 1899-1908 (1999)].

Physicians will commonly recommend endovascular revascularization surgery to treat PAD. Some common surgical interventions target plaque filled arteries by using an inflatable balloon attached to a catheter to compress plaque against the artery wall and restore blood flow to the artery. Unfortunately, 30% of patients require a second intervention within 12 months after the initial surgery due to persistence of symptoms [J. A. Beckman et al., 318261 (2021) Circulation research 128.12 (2021): 1885-1912; J. H. Rogers and J. R. Laird, Circulation. 116(18), 715433 (2007); B. H. Gray, et al., J Vasc Surgery. 25(1), 74-83 (1997)]

Current monitoring methods such as the ankle-brachial index (ABI) and arterial duplex ultrasound (A-DUS) do not provide information on distal perfusion in the foot and are unable to predict a patient's response to surgery prior to the intervention. Additionally, ABI lacks sensitivity for PAD patients with diabetes, who make up 20-40% of the patient population [J. D. Santilli and S. M. Santilli, Am. Fam. Physician. 59(7), 1899-1908 (1999); T. Yoshimura et al., Diabetes Care 29(8), 1884-1890 (2006); D. Xu et al., Vasc. Med. 15(5), 361-369 (2010)]. A novel medical device with improved sensitivity for the assessment of vascular health has the potential to detect PAD earlier, more accurately and for a diverse patient demographic.

Thus, there is a need in the art to develop devices and methods for the assessment of vascular health. The present invention meets this need.

### SUMMARY OF THE INVENTION

Some embodiments of the invention disclosed herein are set forth below, and any combination of these embodiments (or portions thereof) may be made to define another embodiment.

A medical pressure probe device comprises a body having a proximal end and a distal end, with a distal face, at least one optical sensor and at least one light source positioned on the distal face of the body, and at least one force sensor positioned inside the body, and a handle interfaced with the body and movable relative to the body along a path between a neutral position and an engaged position, the handle comprising a spring mechanism that contacts the at least one force sensor when the handle is in the engaged position to measure the applied pressure on the body from the handle, wherein the spring mechanism biases the handle to the neutral position.

In some embodiments, the spring mechanism connects to a central portion of the handle, or to a portion offset from the center of the handle. In some embodiments, the spring mechanism comprises first and second portions connected by a spring, wherein the second portion contacts the at least one force sensor. In some embodiments, the device further comprises a guide positioned in the body configure to align the spring mechanism with the at least one force sensor.

In some embodiments, the at least one optical sensor comprises first, second and third optical sensors, and the at least one light source comprises between 1 and 9 light sources. In some embodiments, the at least one light source is set to at least one wavelength between 500 nm and 950 nm. In some embodiments, the at least one light source comprises first and second light sources, wherein the first light source is set to a wavelength of 780 nm, and the second light source is set to a wavelength of 850 nm. In some embodiments, the at least one light source comprises a third light source, and wherein the first light source is set to a wavelength of 660 nm, the second light source is set to a wavelength of 880 nm, and the third light source is set to a wavelength between 520 nm and 535 nm.

In some embodiments, the at least one optical sensor and at least one light source are arranged in one or more groupings on the distal face of the body. In some embodiments, the one or more groupings comprises 3 groupings arranged in an offset pattern on the distal face of the body. In some embodiments, each grouping comprises a first, second and third light source, and at least one optical sensor, wherein each light source is set to different wavelengths. In some embodiments, the at least one light source is positioned a distance from the at least one optical sensor ranging between 1 mm and 20 mm.

In some embodiments, the body and handle are slidably attached or engaged. In some embodiments, the spring mechanism and the guide comprise similar cross-sectional size and shape to center the spring mechanism in the guide. In some embodiments, a portion of the handle fits over the proximal end of the body. In some embodiments, at least one of the body and the handle comprise retaining means for limiting the travel of the handle relative to the body. In some embodiments, the retaining means comprise end caps, openings, posts, slots, grooves, tabs, alignment features, and combinations thereof.

In some embodiments, the device further comprises a distal peripheral rim at least partially surrounding the distal face of the body. In some embodiments, the device further comprises an adhesive at least partially covering at least one of the distal face and distal peripheral rim.

In some embodiments, the device further comprises a computing system communicatively connected to the sensors and at least one light source, comprising a processor and a non-transitory computer-readable medium with instructions stored thereon, which when executed by a processor, perform steps comprising supplying light via the at least one light source to target site of a subject, capturing measurements from the at least one optical sensor and the at least one force sensor, calculating oxygenated, deoxygenated, and total hemoglobin concentrations and oxygen saturation based on the captured measurements, calculating a pulse amplitude based on the captured measurements, displaying the pulse amplitude, displaying the applied pressure, and recalculating pulse amplitude and hemoglobin concentrations based on the applied pressure.

In some embodiments, the steps comprise displaying the applied pressure with a threshold scale indicating an optimal applied pressure range. In some embodiments, the steps comprise providing feedback to a user if the applied pressure is inside or outside an optimal applied pressure range. In some embodiments, the feedback is provided with an interface for the device, wherein the interface comprises at least one of a speaker, a display, and a graphical user interface (GUI). In some embodiments, the steps comprise calculating a heart rate, and displaying the heart rate.

A pulse amplitude measurement method comprises providing a probe device, positioning the distal face of the body at a target site on a subject, at least partially adhering the distal face to the target site such that the at least one optical sensor and at least one light source are contacting the target site, interfacing the handle with the body of the device, supplying light via the at least one light source, pushing on the handle to the engaged position to apply a pressure to the target site, capturing measurements comprising light intensity response and applied pressure, calculating the oxygenated, deoxygenated, and total hemoglobin concentrations and oxygen saturation based on the captured measurements, calculating a pulse amplitude based on the captured measurements, and displaying the pulse amplitude.

In some embodiments, the steps comprise displaying the applied pressure, recalculating the pulse amplitude and total hemoglobin concentrations based on the applied pressure. In some embodiments, the steps comprise calculating a heart rate, and displaying the heart rate. In some embodiments, the steps comprise displaying the applied pressure with a threshold scale indicating an optimal applied pressure range. In some embodiments, the steps comprise providing feedback to a user of the device if the applied pressure is inside or outside an optimal pressure range. In some embodiments, the feedback is provided with an interface for the device, wherein the interface comprises at least one of a speaker, a display, and a graphical user interface (GUI).

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1D depict an exemplary medical pressure probe device for assessment of vascular health according to aspects of the present invention. FIG. 1A depicts an exemplary probe body (left) and a cutaway view of an exemplary probe handle (right). FIG. 1B depicts a bottom view of an exemplary probe body (left) showing three source-detector modules, each with three LEDs and one photodetector, and (right) a perspective view of a distal portion of a probe body showing openings for a USB-C charging port, as well as reset and bootloader push buttons. FIG. 1C depicts portions of an exemplary probe body comprising a middle portion (left) comprising a ledge for a force sensor and a battery fastener, and a proximal portion (right) comprising a guide cylinder for a spring mechanism, and another fastener for the battery. FIG. 1D depicts a top down view of the inside of an exemplary probe body with a force sensor and battery secured in place. FIG. 1E depicts an exemplary circuit board with microcontroller that is positioned within the body of the device. FIG. 1F depicts an exemplary probe device comprising a probe handle (left) and a probe body (right).

FIG. 2A through FIG. 2F depict another exemplary probe device for the assessment of vascular health. FIG. 2A depicts an exemplary probe cap or handle (left), and an exemplary probe body (right). FIG. 2B depicts a bottom perspective view of an exemplary probe device showing two light sources and a detector. FIG. 2C depicts a side view of an exemplary probe cap and probe body. FIG. 2D depicts a side view of an exemplary probe handle positioned in a proximal position relative to a probe body. FIG. 2E depicts a side view of a probe handle positioned in a distal position relative to a probe body. FIG. 2F depicts an enlarged view of the proximal end of a probe body comprising a slot that guides and retains a portion of the handle.

FIG. 3A is a diagram of an exemplary graphical user interface (GUI).

FIG. 3B is a diagram of an exemplary computing device.

FIG. 4A shows results from the use of a medical pressure probe device showing the percent change of total hemoglobin [%] in response to a change in applied pressure [N/mm²] for ten tests with healthy subjects. FIG. 4A displays the percent change of total hemoglobin [%] in response to a change in applied pressure [N/mm²] for 3 healthy volunteers at the medial plantar (left), dorsalis pedis (center), and posterior tibial (right) angiosomes. A negative change in pressure [N/mm²] corresponds to a decrease in applied pressure from the initial time point. A negative percent change in HbT [%] indicates a decrease in HbT [uM] from the initial time point. FIG. 4B shows results from the use of a medical pressure probe device showing the percent change of total hemoglobin [%] at each change in applied force for ten tests with healthy subjects at the same locations as in FIG. 4A. P01—change from initial/baseline to first application of force; P12—change from first to second application of force; P23—change from second to third application of force; P34—change from third to fourth application of force; P4E—change from fourth application of force back to baseline.

FIG. 5A through FIG. 5D show results from the use of an exemplary probe device on various subjects. FIG. 5A is a plot showing signal [V] for the 780 nm (cyan) and 850 nm (orange) wavelength laser diodes and pressure [N/mm²] (yellow) vs time [sec]. FIG. 5B is a plot showing HbT [uM] (black) and pressure [N/mm²] (red) vs time [sec] for a representative healthy volunteer at the posterior tibial tissue region. FIG. 5C is a plot showing HbT [uM] (black) and pressure [N/mm²] (red) vs time [sec] for a representative healthy volunteer at the posterior tibial tissue region. FIG. 5D is a plot showing HbT [uM] (black) and pressure [N/mm²] (red) vs time [sec] for a representative healthy volunteer at the posterior tibial tissue region.

FIG. 6A is a set of plots showing the average pulse peak to peak amplitude [uM] vs pressure [N/mm²] for three healthy volunteers with measurements taken at the dorsalis pedis, medial plantar, and posterior tibial tissue regions. FIG. 6B is a plot and image displaying the expected behavior of total hemoglobin [uM] in response to applied pressure [N/mm²] (top) for PAD patients who respond to treatment (positive outcome) and those who do not (negative outcome), and an effect of pressure on tissue (bottom).

FIG. 7A is a plot displaying Signal [V] for the 780 nm (green) and 850 nm (cyan) wavelength laser diodes and pressure [N/mm²] (orange) vs time [sec]. FIG. 7B is a plot displaying the corresponding HbT [uM] (blue) and pressure [N/mm²] (orange) vs time [sec] from a representative heathy volunteer test.

### DETAILED DESCRIPTION

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Medical Device for Assessment of Vascular Health

The present invention relates to a medical pressure probe device for the assessment of vascular health. For example, in some embodiments the probe device is used to assess peripheral arterial disease (PAD). The disclosed probe device is configured to be grasped with one or more hands, with a distal portion of the probe contacting the skin of a subject over a target area. The device provides reliable and accurate means for assessing vascular health using a force sensor that ensures proper usage. In some embodiments, while a distal portion of the probe is held against target vasculature of a subject, sensor measurements are captured and displayed in real-time, providing feedback to a user if correct force or pressure is being applied to the probe.

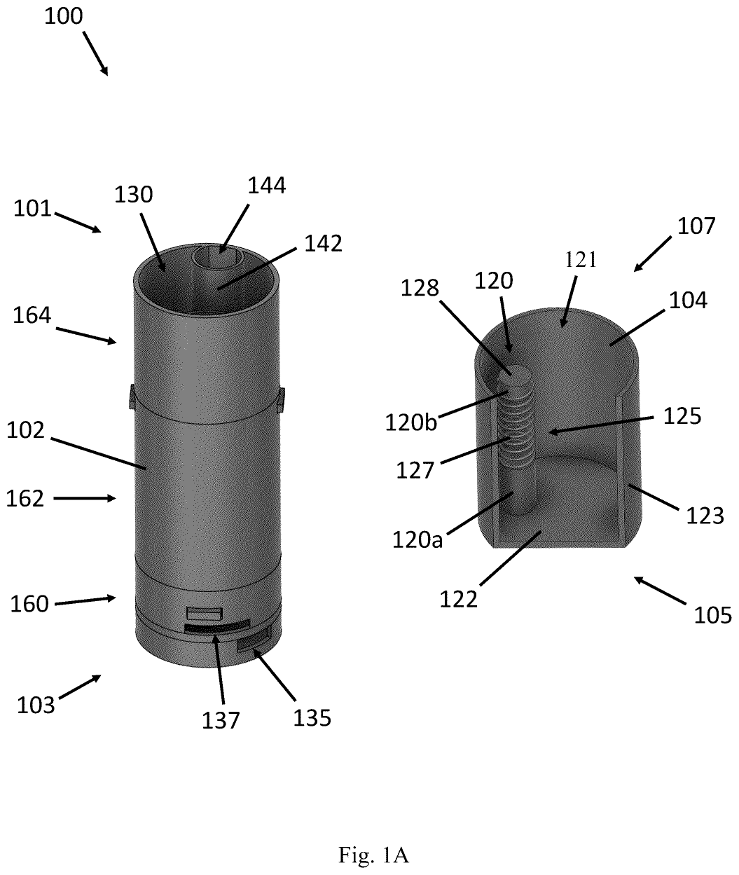

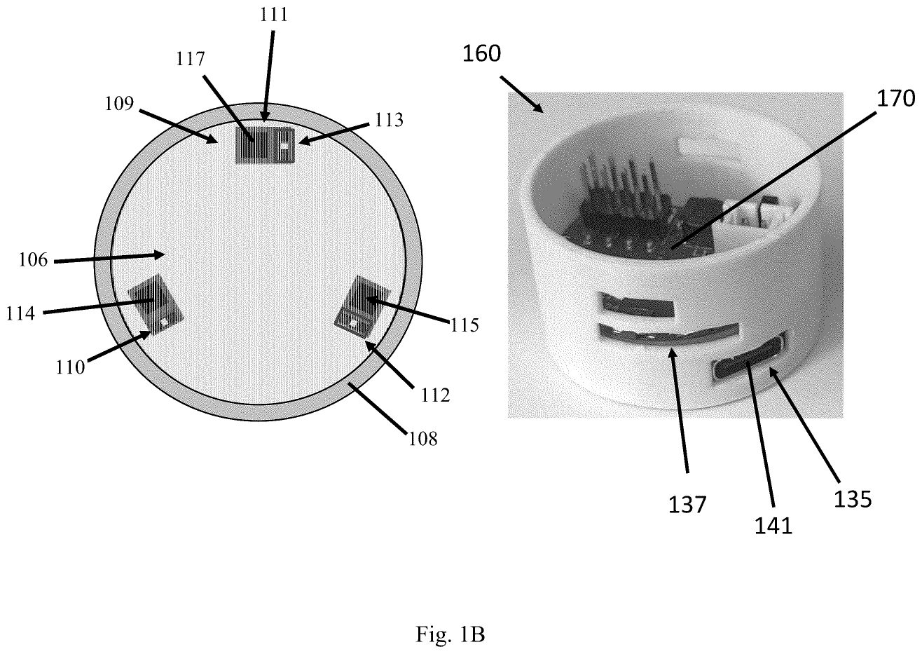

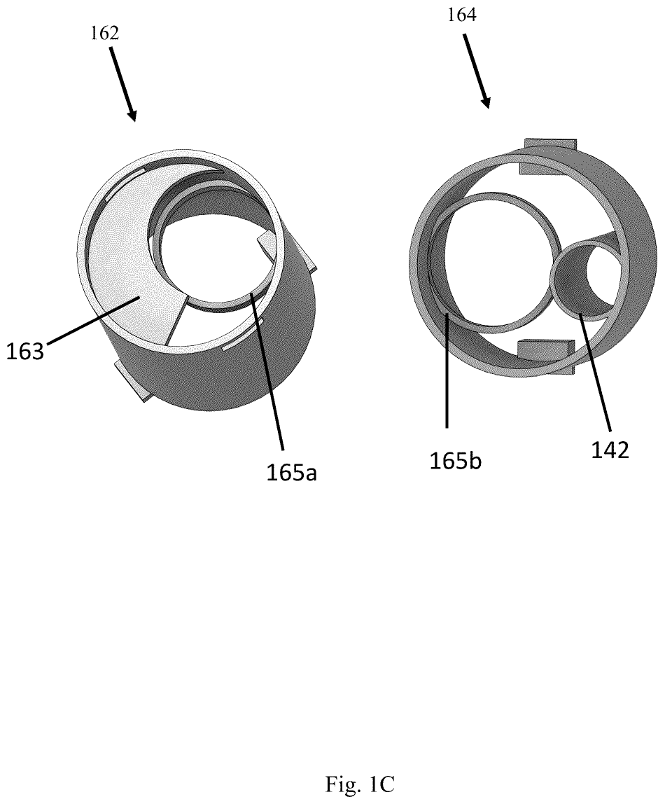

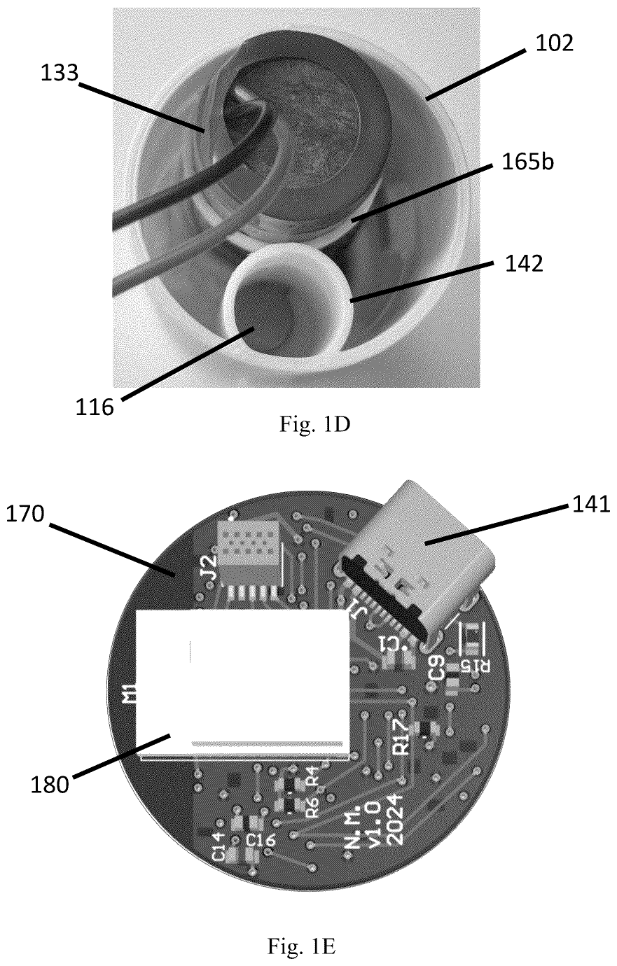

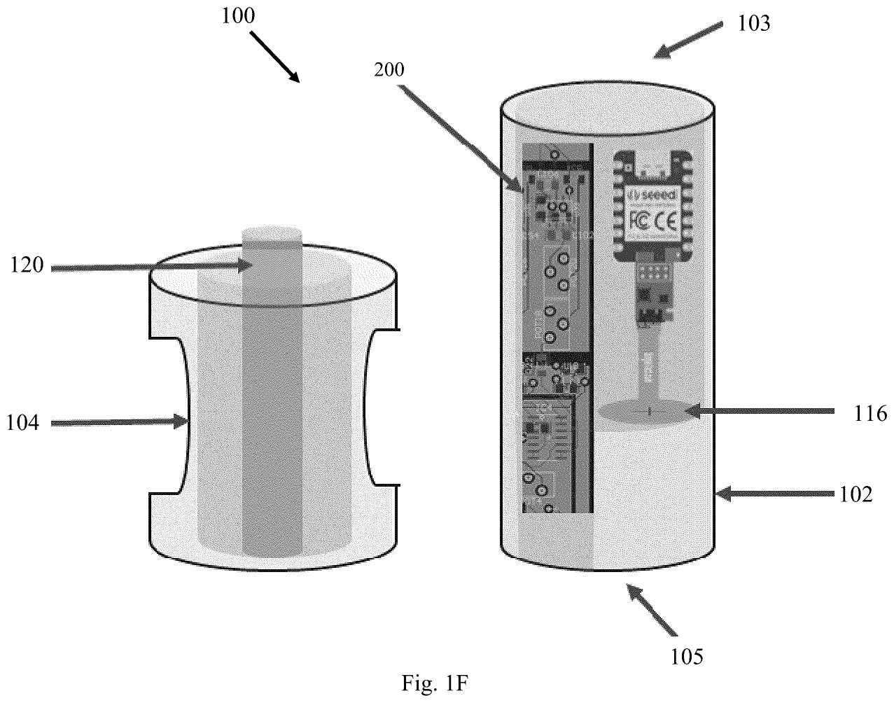

Referring now to FIGS. 1A-1E, depicted is an exemplary medical pressure probe device 100 for the assessment of vascular health. Generally, device 100 comprises a body 102 and a handle 104 that interfaces with body 102. While device 100 is being used on a subject, handle 104 interfaces with body 102, and a distal portion of handle 104 creates pressure or force on a force sensor positioned inside body 102. Generally, body 102 is an elongate hollow body or tube having a proximal end 101 and a distal end 103, with a distal face 106 configured to make contact with a target site or area of interest on a subject. In some embodiments, body 102 is a cylinder or tube having a circular, ovular, square, rectangular, triangular, or polygonal shaped cross section. Body 102 comprises a distal face 106 positioned at distal end 103 which provides a surface for positioning one or more light sources, sensors, and/or sensor groupings.

Handle 104 generally serves as a grasping portion for the device. In some embodiments, handle 104 is an elongate hollow body or tube having a proximal end 105 and a distal end 107, and is configured to interface with body 102 and a force sensor thereof. In some embodiments, handle 104 is a cylinder or tube having a proximal end cap 122 with circular, ovular, square, rectangular, triangular, or polygonal shaped cross section. Handle 104 generally comprises sidewalls 123 with an opening 121, with a member 120 extending up from proximal end cap 122 for interfacing with a force sensor of body 102. In some embodiments, a portion of handle 104 fits over proximal end 101 of body 102, wherein the diameter of the body is similar to an inner diameter of the opening 121 of the handle. This can function as a compression fit, or a slightly loose fit, to ensure the handle is retained substantially coaxial with the body when the device is used.

The member 120 may be formed in any size and shape and can extend up from proximal end cap 122 any distance. For example, in some embodiments, member 120 is a circular post or prong that extends up from proximal end cap 122 a height similar to sidewalls 123. A cross-sectional shape of member 120 may include any of circular, ovular, square, rectangular, triangular, or polygonal. Member 120 may extend up from any position on proximal end cap 122, such as from the center, or offset from center. In some embodiments, member 120 comprises one or more spring mechanisms 125. In some embodiments, the spring mechanism 125 is comprised of a proximal portion 120a of member 120 connected at least partially by a spring 127 to a distal portion 120b. The spring 127 compresses when the two portions are pushed together or compressed. In some embodiments, proximal portion 120a and distal portion 120b are slidably attached and may comprise retaining means to limit the travel of distal portion 120b, and/or set a preload on spring 127. In some embodiments, proximal portion 120a and distal portion 120b are sized and shaped to fit partially inside of spring 127, and may function as spring isolators. In some embodiments, the spring is a compression spring with an outer diameter between 8 mm and 10 mm, an inner diameter between 6 mm and 9 mm, a free length between 10 mm and 50 mm, and a maximum deflection between 30 mm and 35 mm. The spring may have a spring constant in the 0.3 N/mm to 0.5 N/mm range. In some embodiments, the maximum load of the spring is within 10 N and 100 N.

Although an exemplary spring mechanism 125 with spring 127 is shown, it should be appreciated that any disclosed member 120 may further comprise a spring integrated into the member and/or at least partially surrounding the member to bias handle 104 to a neutral position when pressure is not actively applied by the user and create slight resistance when handle 104 is applying pressure to body 102. In some embodiments, spring 127 acts as a buffer to smooth out the captured force sensor measurements.

In some embodiments, the spring (e.g., spring 127) of member 120 and/or spring mechanism 125 makes it easier to control increases in the amount of pressure applied to a subject (e.g., to the tissue of the subject), makes it easier to release pressure and/or return to a baseline, and makes it easier to standardize the range of pressures or forces exerted on each individual subject. In some embodiments, a spring or spring mechanism returns handle 104 to a neutral position when pressure is not actively applied by the user. In some embodiments, handle 104 is neutral in a proximal position resting over body 102, and moves to a distal engaged position when force is applied by the user, where a force measurement may be taken.

Generally the force sensor measures the pressure or force exerted on body 102 by handle 104. For example, in some embodiments, handle 104 is configured to engage body 102, move relative to body 102, and exert force on a member 120 that contacts the force sensor. In some embodiments, the handle 104 exerts force on the body 102 via the spring mechanism 125. In some embodiments, the distal portion 120b of spring mechanism 125 contacts a force sensor and exerts variable amounts of force on body 102 relative to the force exerted on the spring mechanism 125 by handle 104.

Aspects of the present invention relate to an exemplary body 102 for device 100. Body 102 may be formed from one or more portions, or may be formed as a single piece or unit. In some embodiments, the portions of body 102 are removably attached together. Shown in FIG. 1B (right) is a distal portion 160 for body 102. Referring now to FIG. 1C, shown is a middle portion 162 and a proximal portion 164. In some embodiments, middle portion 162 comprises a ledge 163 for a force sensor, and first fastener 165a for retaining a battery. In some embodiments, the proximal portion 164 comprises a member guide 142 (e.g., guide cylinder) for a spring mechanism 125 or member 120, and a second fastener 165b. In some embodiments, body 102 comprises one or more fasteners to hold the battery 133 in place inside body 102. Body 102 may comprise between 1 and 5 fasteners for the battery 133. The exemplary probe device 100 shown in FIG. 1D comprises two fasteners (second fastener 165b shown) attached to internal positions of body 102.

In some embodiments, body 102 comprises a member guide 142 (e.g., an elongate hollow cylinder) comprising an opening 144 on proximal end 101 configured as a guide for member 120 and/or spring mechanism 125 of handle 104. The member guide 142 comprises a force sensor positioned therein, wherein member 120 and/or spring mechanism 125 make contact with the force sensor when body 102 and handle 104 are interfaced and in the engaged position. The member guide 142 may be formed in any size and shape, for example member guide 142 may be similarly sized and shaped to member 120 and/or spring mechanism 125. There can be any number of member 120, spring mechanism 125 and/or member guide 142. For example, in some embodiments, there are two spring mechanism 125 connected to handle 104 that are positioned in diametrically opposed positions. In some embodiments, there are 1, 2, 3, 4 5 member 120 and/or spring mechanism 125 positioned in any arrangement. It should be appreciated that for each member 120 and/or spring mechanism 125 there is a corresponding member guide 142 in body 102.

In some embodiments, device 100 comprises a battery 133 (e.g., a lithium-ion battery) positioned inside body 102 for powering the device. Body 102 may comprise any number of openings or slits for charging and controlling the device. For example, in some embodiments, distal portion 160 of body 102 comprises an opening 135 configured to house a charging port 141 (e.g., a USB Type-C charging port). In some embodiments, distal portion 160 of body 102 comprises an opening 137 configured for positioning one or more buttons for controlling the device. For example, opening 137 may be configured for accessing reset and bootloader push buttons for configuring a computing device (e.g., a microcontroller 180) of device 100. Referring to FIG. 1E, shown is an exemplary circuit board 170 sized and shaped to fit within body 102, comprising a microcontroller 180 and charging port 141.

In some embodiments, body 102 has an outer diameter ranging between 10 mm and 100 mm, 15 mm and 90 mm, 15 mm and 80 mm, or 20 mm and 40 mm. In some embodiments, body 102 has an inner diameter ranging between 1 mm and 100 mm, 6 mm and 100 mm, or 10 mm and 40 mm. In some embodiments, body 102 has a length ranging between 50 mm and 200 mm. In some embodiments, member guide 142 comprises a width or diameter ranging between 0.1 mm and 50 mm, and a height ranging between 10 mm and 140 mm. In some embodiments, distal face 106 and/or peripheral rim have diameters ranging between 5 mm and 100 mm, or 20 mm and 80 mm, or 30 mm and 70 mm.

In some embodiments, handle 104 has an outer diameter ranging between 10 mm and 100 mm, 15 mm and 90 mm, 15 mm and 80 mm, or 20 mm and 40 mm. In some embodiments, handle 104 has an inner diameter ranging between 10 mm and 60 mm. In some embodiments, handle 104 has a length ranging between 50 mm and 200 mm. In some embodiments, member 120 and/or spring mechanism 125 comprise a width or diameter ranging between 0.1 mm and 50 mm, and a height ranging between 10 mm and 140 mm, or a height of about 80 mm.

Body 102 and/or handle 104 may be formed from any suitably rigid materials. Body 102 and/or handle 104 may comprise any of a 3D printed material, a metal, a plastic, and any combinations thereof. In some embodiments, body 102 and/or handle 104 comprise at least one of PLA, PEEK, ABS, PET, PVA, polycarbonate, polypropylene, polyethylene, polyamide, and polyvinyl chloride. In some embodiments, body 102 and/or handle 104 comprises at least one biocompatible material and/or coating.

Certain aspects of device 100 (e.g., body 102 and/or handle 104) may be made using an additive manufacturing (AM) process. Among the most common forms of additive manufacturing are the various techniques that fall under the umbrella of “3D Printing”, including but not limited to stereolithography (SLA), digital light processing (DLP), fused deposition modelling (FDM), selective laser sintering (SLS), selective laser melting (SLM), electronic beam melting (EBM), and laminated object manufacturing (LOM). These methods variously “build” a three-dimensional physical model of a part, one layer at a time, providing significant efficiencies in rapid prototyping and small-batch manufacturing. AM also makes possible the manufacture of parts with features that conventional subtractive manufacturing techniques (for example CNC milling) are unable to create.

Suitable materials for use in AM processes include, but are not limited to, nylon, polyethylene terephthalate (PET), acrylonitrile butadiene styrene (ABS), resin, polylactic acid (PLA), polystyrene, and the like. In some embodiments, an AM process may comprise building a three-dimensional physical model from a single material, while in other embodiments, a single AM process may be configured to build the three-dimensional physical model from more than one material at the same time.

Generally, device 100 comprises one or more sensors 109 (e.g., optical sensors) positioned at distal end 103 of body 102, and at least one force sensor (e.g., force sensor 116) positioned at least partially inside of body 102 configured to measure force from handle 104. In some embodiments, device 100 further comprises one or more sensors 109 positioned on distal face 106. In some embodiments, sensors 109 may comprise any known optical sensors or photodetectors. In some embodiments, the sensors 109 comprise any of the sensors positioned at the distal end of body 102 and/or partially inside the distal end of body 102 as disclosed herein. In some embodiments, sensors 109 comprise any of sensors 465 described herein for a computer 400.

The at least one force sensor can be any suitable sensor that can measure force and/or pressure including capacitive sensors, pressure sensors, strain sensors, force transducers, load cells, and the like. In some embodiments, each force sensor has a height, and width or diameter ranging between 1 mm and 30 mm, or 4 mm and 20 mm, or 6 mm and 15 mm. In some embodiments, each force sensor has an area between 10 mm² and 200 mm². In some embodiments, the force sensor is a circular, capacitive sensor with a load between 1 N to 100 N, a diameter of 8 mm, a width of 0.3 mm, and a sensor response time under 1 ms.

Device 100 may comprise any number of light sources. In some embodiments, device 100 comprises at least one light source positioned on distal face 106. The sensors 109 and light sources can be positioned together in any arrangement, such as in groupings or modules, or separately placed, as discussed further herein. For example, in some embodiments, device 100 comprises at least one light source and at least one sensor 109 (e.g., optical sensor, photodetector) positioned on distal face 106 of body 102.

Referring now to FIG. 1B, in some embodiments, device 100 comprises at least a first light source 110, a second light source 112, and at least one sensor 114. In some embodiments, device 100 comprises a third light source 113. In some embodiments, the at least one light source comprises any number of laser diodes and/or LEDs, wherein each light source produces a specific wavelength, or spectrum of light. In some embodiments, device 100 comprises a second sensor 115 and a third sensor 117. In some embodiments, device 100 comprises 4 light sources, 2 sensors (e.g., photodetectors), and one force sensor positioned near the light sources and photodetectors. In some embodiments, device 100 comprises between 1 and 20 light sources and between 1 and 20 sensors, and between 1 and 10 force sensors. For example, in some embodiments, device 100 comprises between 6 and 9 light sources, and 2 or 3 sensors.

In some embodiments, the light sources and sensors are positioned on the distal end 103 of the probe body in any pattern or arrangement, including equilateral, offset, circular, radial and/or circumferential patterns. Referring again to FIG. 1B, shown in this arrangement are a plurality of light source/sensor module 111 positioned in an offset pattern on distal face 106. In some embodiments, each light source/sensor module 111 is positioned near the edge of the distal face 106. In some embodiments, each light source/sensor module 111 comprises a plurality of light sources (e.g., 3 light sources) and at least one sensor (e.g., photodetector).

In some embodiments, the at least one light source comprises a bulb, LED, laser diode, and the like, and any combinations thereof. In some embodiments, the at least one light source comprises 5 mW 5.6 mm-diameter laser diodes. In some embodiments, the at least one light source comprises three 0.5 mm-diameter LEDs. It should be appreciated that the at least one light source (and at least one sensor) may comprise any source/photodetector module, optical data acquisition system, optical sensing module, optical bio-sensor, or the like, known by one of ordinary level of skill in the art. For example, the at least one light source and at least one sensor may comprise an integrated optical module, such as the source detector module MAXM86161 from Analog Devices. In some embodiments, at least one light source has a width or diameter between 0.1 mm and 15 mm, between 0.5 mm and 12 mm, between 3 mm and 10 mm, between 0.1 mm and 0.9 mm, or between 0.25 mm and 0.75 mm, or has a width or diameter of about 0.25 mm, 0.5 mm, or 0.75 mm. In some embodiments, the at least one light source has a diameter of about 0.5 mm.

In some embodiments, the at least one light source is set to produce one or more wavelengths between 500 nm and 950 nm. In some embodiments, the at least one light source is set to a wavelength of 780 nm, and 850 nm, respectively, or 660 nm and 880 nm, or between 520 nm and 535 nm. In some embodiments, the at least one light source has a wavelength selected from 880 nm, 660 nm, 520 nm, 535 nm, 670 nm, 780 nm, 808 nm, 850 nm, and 904 nm, or any wavelength between 520 nm and 910 nm, or between 520-535 nm. In alternative embodiments, a different set of wavelengths may be used in the green, red, or near-infrared light spectra. These wavelengths provide a range of spectral information to reconstruct the absorption coefficient, the reduced scattering coefficient, oxygen saturation, oxygenated hemoglobin ([HbO₂]), and/or deoxygenated hemoglobin ([Hb]) while working within the limited selection of wavelengths commercially available. In some embodiments, the at least one light source has radiant power between 5 mW and 10 mW. In some embodiments, the at least one light source has a radiant power between 5 mW and 40 mW.

In some embodiments, the at least one light source is positioned between 1 mm and 20 mm away from at least one photodetector. In some embodiments, first light source 110 and second light source 112 are positioned at least 20 mm away from sensor 114 on distal face 106. In some embodiments, first light source 110 and second light source 112 are positioned ranging from 1 mm to 20 mm, 2 to 15 mm, or 2 mm to 10 mm, from sensor 114 on distal face 106.

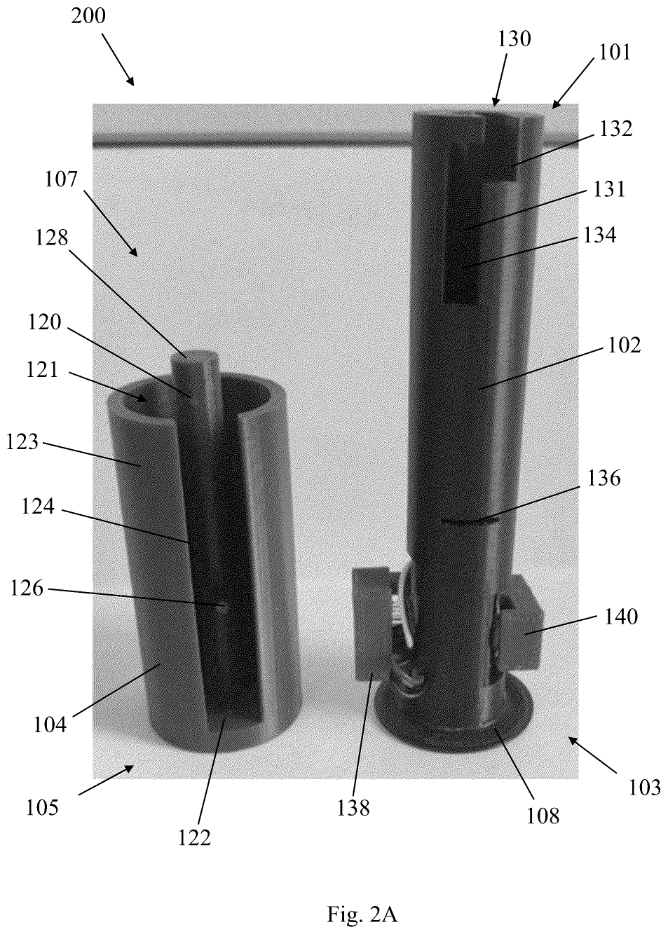

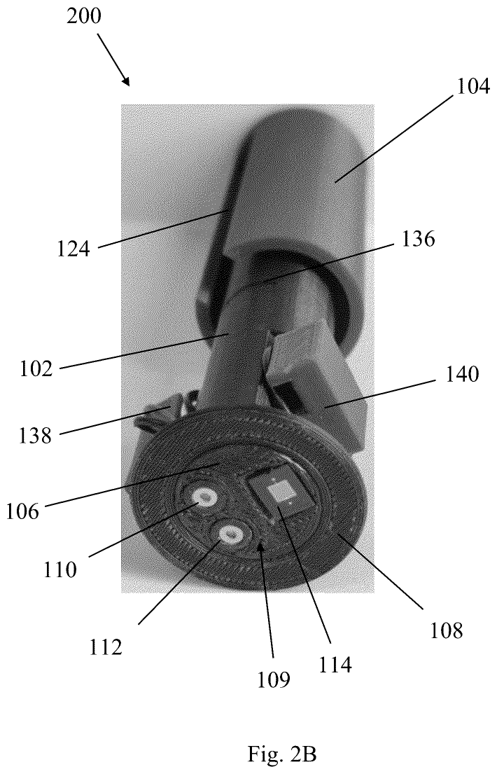

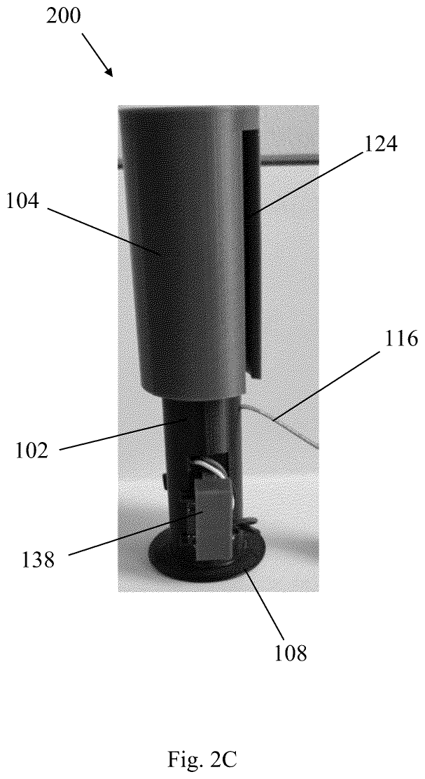

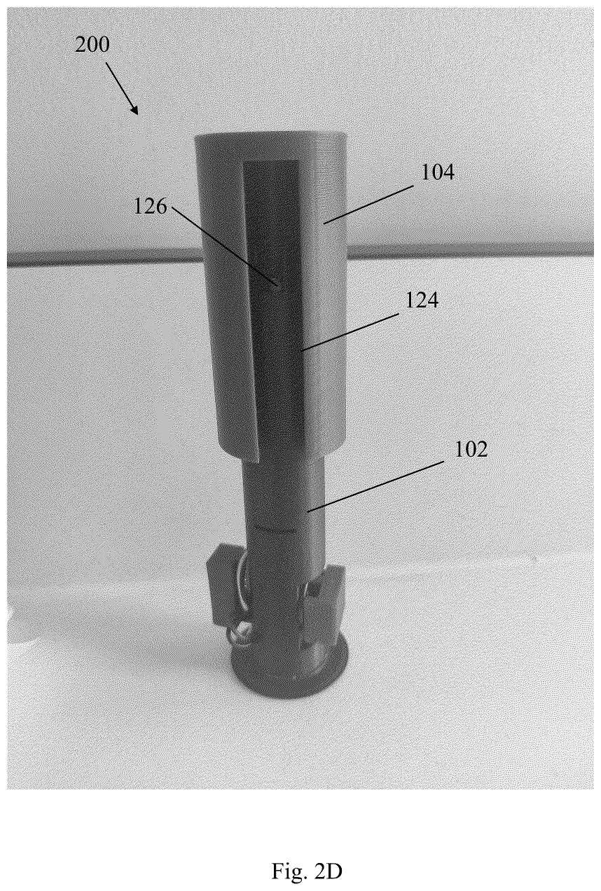

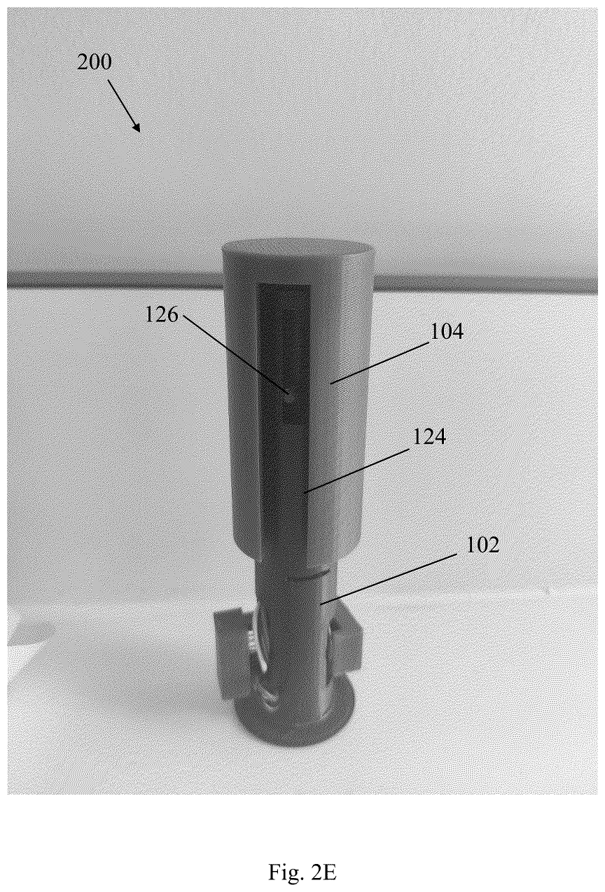

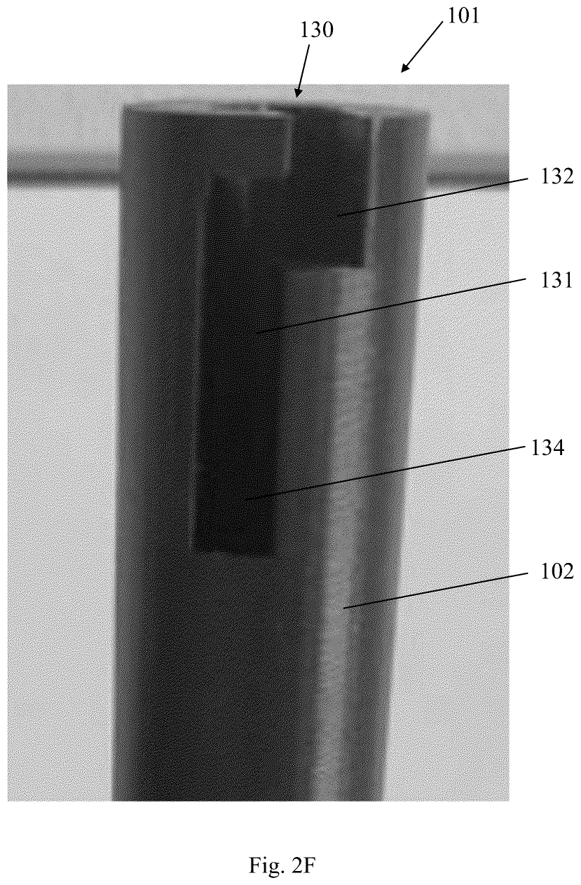

An exemplary embodiment of a probe device is shown in FIG. 1F, and another embodiment of a probe device is disclosed in FIGS. 2A-2F and discussed herein. It should be appreciated that the exemplary probe device of FIGS. 2A-2F may share any of the features of the probe devices of FIGS. 1A-1F, and vice versa.

Referring now to FIG. 2A, depicted is a handle 104 (left) and body 102 (right) for an exemplary device 200. In some embodiments, the body of handle 104 comprises a member 120 extending from a proximal end cap 122. Handle 104 comprises one or more sidewalls 123 extending up from proximal end cap 122 and a side opening 124 in the sidewalls connected with opening 121. Generally member 120 extends upwards a length or distance and terminates in a distal face 128 that is planar. In some embodiments, member 120 comprises a lateral pin or tab 126 positioned along its length and aligned with side opening 124. Member 120 can extend up and terminate at any length relative to the sidewalls 123 and opening 121, such as extending partially out of opening 121.

In some embodiments, handle 104 is configured to engage with and move relative to body 102 and reside in a range of positions for measuring force. Referring now to FIG. 2D, shown is an exemplary device 100 wherein handle 104 resides in a proximal position relative to body 102. Referring to FIG. 2E, shown is an exemplary device 100 wherein handle 104 is placed in a distal position relative to body 102, and a portion of handle 104 contacts a force sensor in body 102. In some embodiments, distal face 128 of handle 104 contacts a force sensor when handle 104 is in the distal position, and measures the pressure or force between handle 104 and body 102.

In some embodiments, body 102 comprises a proximal opening 130 and a slot 131 extending down at least a portion of body 102. In some embodiments, the slot 131 comprises a lateral opening through the side of body 102, extending distally from proximal opening 130 along at least a portion of body 102. The slot 131 is configured as a guide or index to orient handle 104 into body 102. In some embodiments, a portion of handle 104 releasably engages slot 131 when the body 102 and handle 104 are interfaced. For example, in some embodiments, lateral tab 126 of handle 104 engages slot 131 of body 102 in order to direct the handle 104 along at least a first and second path. In some embodiments, slot 131 comprises a first region 132 which is stepped to a second region 134. The handle 104 is configured to rotate and then translate distally to move from first region 132 to second region 134. When handle 104 is in the distal position, lateral tab 126 slides within second region 134 and a force measurement can be taken.

Referring now to FIG. 2B, in some embodiments, body 102 further comprises a distal peripheral rim 108 at least partially surrounding distal face 106. In some embodiments, distal face 106 and/or distal peripheral rim 108 comprise at least one adhesive material, layer and/or coating at least partially covering the face and/or rim. In some embodiments, the adhesive material is Mylan. In some embodiments, the adhesive material is Tegaderm film. The adhesive material or coating may comprise any sterile, medical grade adhesive or biocompatible adhesive as would be known by one of ordinary level of skill in the art.

In some embodiments, body 102 further comprises a lateral slit 136 extending through body 102, wherein a portion of a force sensor 116 extends through lateral slit 136 and electrically connects to a computing device. Although an exemplary wired connection for force sensor 116 is shown in FIG. 2C, it should be appreciated that force sensor 116 may connect to a computing device internal to the probe device, or wirelessly to an external computing device, wherein force sensor 116 resides entirely within body 102 and does not extend through lateral slit 136.

In some embodiments, device 200 comprises one or more housings for encasing controllers, computers, sensors, wires or the like. Referring back to FIG. 2A, in some embodiments device 200 further comprises at least a first housing 138 and second housing 140 attached to body 102 configured to house circuitry, controllers, sensors wiring, electrical connections, computing devices, sensors, and the like. In some embodiments, first housing 138 and second housing 140 comprise one or more electrical connections between the sensors and light sources of device 200 and one or more computing devices. An exemplary device is shown with electrical housings and connections to an external computing device, however it should be understood that a computing device may also be positioned within body 102 without the need for the separate housings (as shown in FIG. 1F). In some embodiments, the disclosed devices wirelessly connect to an additional computing device that provides an interface for the device. In some embodiments, any of the disclosed devices comprise one or more displays or screens, for example an LCD screen integrated into body 102 and/or handle 104. In some embodiments, a wireless (e.g., Bluetooth enabled) computing device is stored inside body 102 of the probe device, and can wirelessly connect to an external computing device for producing a user interface (UI).

The disclosed probe devices are configured to measure the pressure or force applied from the probe device on the subject (i.e., the tissue interface pressure (TIP)) while also measuring biometrics of the area or region of interest on the subject. To measure pressure applied from the probe device on a subject, a portion of handle 104 extends into body 102 and contacts a force sensor to measure the force applied from handle 104 on body 102, which can be used to calculate the force or pressure on the subject. Particularly, in some embodiments handle 104 comprises a member 120 and/or spring mechanism 125 with a planar distal face 128 that contacts a force sensor positioned inside body 102 creating a perpendicular pressure on the sensor. This measured value can be used to calculate the TIP, as discussed further herein.

In some embodiments, the disclosed probe devices are configured for comfortable placement against a person's skin with the at least one light source and at least one sensor on distal face 106 flush with the tissue surface. Light from the at least one light source is directed into the person's body such that light is reflected back and is detected by the at least one sensor (e.g., optical sensor). In some embodiments, device 100 enables dynamic vascular optical spectroscopy with pressure sensing to monitor the relationship between applied pressure or force and blood volume changes in tissues of interest.

The disclosed devices function with at least one light source positioned on distal face 106 that shines light (e.g., visible light, non-visible light, deep red, near-infrared, red light, green light) into the tissue under investigation. That light is absorbed and scattered by the tissue, and any light emitted is collected by a sensor (e.g., an optical sensor, a photodetector) that is in contact with the tissue surface. Simultaneously, force data representing the force or pressure that distal face 106 exerts on the subject's tissue surface is collected from at least one force sensor. Data representing the detected light and pressure is then forwarded from the sensors of the device to a computing device (e.g., a microcontroller 180 and/or a computer 400 discussed herein). In some embodiments, this data is then used to generate maps of the changes in the concentrations of oxy-hemoglobin [HbO₂], deoxy-hemoglobin [Hb], total hemoglobin [HbT]=[HbO₂]+[Hb], and/or oxygen saturation StO₂=[HbO₂]/[HbT], and correlates this data to the collected force data, in the region corresponding to the body part under investigation. In some embodiments, other tissue parameters, such as water concentration [H₂O], lipid concentration, and/or tissue scattering are derived. In some embodiments, traces and maps of the disclosed parameters can be displayed in real time on a monitor or display via a graphical user interface (GUI) discussed further herein. In some embodiments, blood circulation and/or blood perfusion data is recorded and/or plotted as a function of the applied pressure or force. Blood circulation and/or blood perfusion data may comprise blood volume (i.e., the quantity of blood in the tissue region being sensed), the blood flow (i.e., the rate of flow of blood in the tissue region being sensed), a pulsatile signal (i.e., the change in blood volume), change in blood flow, and/or change in blood pressure in the tissue region being sensed.

Disclosed herein is a GUI that functions to operate any disclosed probe device and view measurements and results of the disclosed methods. The GUI is configured to display imaging, data and results of the disclosed device for real-time data analysis and prediction. This includes any measurements, imaging, live-imaging, subject data, and associated results as produced by the disclosed devices and methods. In some embodiments, the GUI enables predictive analytics such as disease diagnosis and feedback to a user. The GUI is produced from a software operating on the probe device and/or a peripheral device (e.g., a computing device, a handheld device). In some embodiments, the GUI is produced on a display integrated into the probe device. In some embodiments, users can start and stop data acquisition through the GUI.

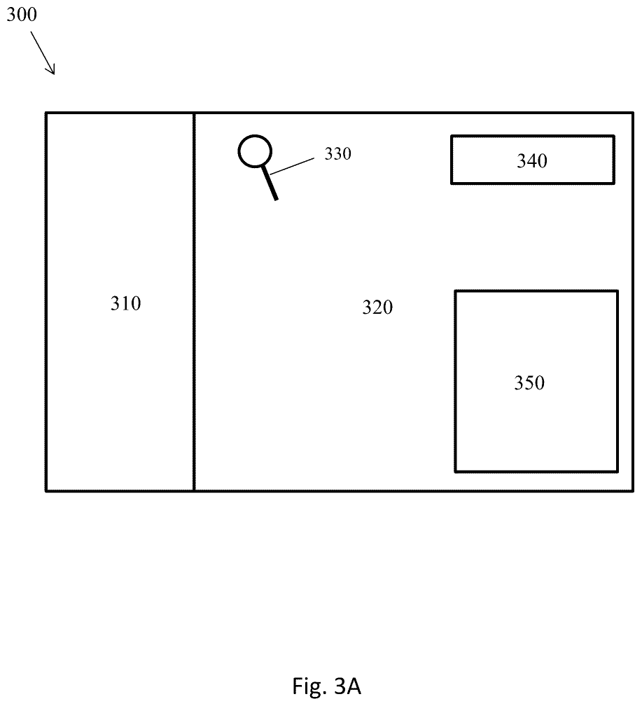

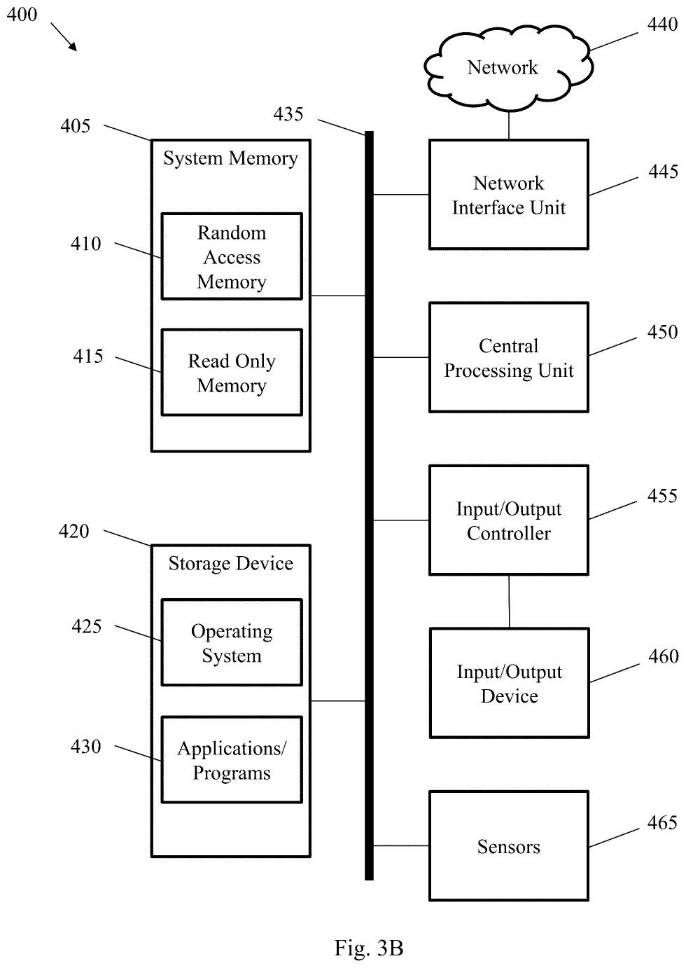

Referring now to FIG. 3A, shown is a diagram of an exemplary GUI comprising one or more screens or dashboards. Depicted is an exemplary screen or dashboard 300 for the GUI comprising a navigation bar 310 and content area 320. In some embodiments, dashboard 300 further comprises a search tool 330 and drop down menu or selector 340 (e.g., for profiles, content and/or analytics). In some embodiments, dashboard 300 provides one or more tabs and/or lists with organized widgets, modules, alerts, graphs, and/or reports displayed in the content area 320 that provides sensor data, plots, subject-related data and other metrics and results. In some embodiments, dashboard 300 provides means for configuring and viewing one or more profiles related to a subject. In some embodiments, content area 320 displays data and analytics for a profile related to each subject. In some embodiments, the GUI provides instructions, notifications and alerts relating to the use of any disclosed probe device.

In some embodiments, the GUI displays measurements and results of the device in real time to a user. In some embodiments, data from the sensors (e.g., photodetectors) and force sensor are displayed in real-time on the GUI. In some embodiments, the force sensor continuously records applied force and sends information to GUI to be displayed. In some embodiments, the force exerted on the probe is read by the force sensor and displayed as pressure [N/mm²] in real-time. In some embodiments, optical data from the sensors (e.g., photodetectors) are simultaneously acquired by the probe device and displayed in real-time on the GUI. In some embodiments, the GUI displays both the signals from the sensors and force sensor readings on two adjacent graphs in real-time.

In some embodiments, the users can store and analyze data using the GUI, including reconstructing data to calculate the oxygenated, deoxygenated, and total hemoglobin concentrations and oxygen saturation, and information regarding pulse amplitude versus applied pressure. In some embodiments, to ensure that a constant force is applied while using the device, the GUI provides the user with visual and/or audible feedback on changes in applied force throughout the acquisition process. In some embodiments, the feedback comprises a threshold scale, color-coded scale or graph indicating the correct or optimal range of applied pressure or force. Any information shown in the GUI may be uploaded to a portal or synced with a database.

Aspects of the present invention relate to exemplary methods of use for the disclosed probe devices. In some embodiments, the disclosed devices are configured to measure, assess and/or monitor vascular health of a subject. In some embodiments, the probe device (along with the disclosed GUI) is configured to diagnose and predict vascular disease in a subject. In some embodiments, the probe device is configured to predict patient outcomes to treatment before they undergo a surgical intervention. The probe devices are configured as a handheld device, wherein a user positions at least a portion of probe device in contact with a subject to assess and/or monitor vascular health. In some embodiment, the probe device may be used for intraoperative monitoring during vascular surgery to improve the efficacy of arterial revascularization for patients suffering from PAD. The handheld design of probe device permits a user (e.g., a surgeon) to view changes during surgery and to help determine the efficacy of revascularization procedures. In some embodiments, the probe device measures and records blood circulation as a function of the applied force. In some embodiments, the prober device measures pulse amplitude, water concentration, and/or lipid concentration in a subject.

Aspects of the present invention relate to methods of assessing vascular health in a subject. An exemplary method of assessing vascular health comprises the steps of providing a probe device comprising a light source, photodetector and force sensor, positioning the probe device on a subject, supplying light via at least one light source to a portion of the subject, capturing measurements or collecting signals from the photodetector and force sensor, calculating a pulse amplitude based on the collected signals, and displaying the pulse amplitude. In some embodiments, the step of collecting signals comprises detecting reflected light with the photodetector and detecting force with the force sensor.

Another exemplary method for pulse amplitude measurement comprises the steps of providing any probe device (e.g., device 100, 200) of the present invention, positioning the sensors at a target site on a subject, inserting the handle into the body of the device, supplying light via at least one light source, pushing on the handle in a distal direction to apply a force to the target site, collecting signals comprising a measured light response and a force applied from the sensors, displaying the reflected light intensity signals and applied force measurements in real time during data acquisition, converting the collected light signals into oxy, deoxy, and total hemoglobin concentration and oxygen saturation based on mathematical light diffusion models, calculating a pulse amplitude based on the collected signals, displaying the pulse amplitude, determining the relationship between pulse amplitude and applied force, and calculating the change in hemoglobin for each change in applied force. Any disclosed method further comprises the step of measuring heart rate, and displaying heart rate using device 100. Any disclosed measurements, calculations, and results can be displayed on a GUI of device 100.

The force or pressure applied from the body to the handle influences the readings or measurements captured by the disclosed probe devices. For example, in some embodiments, a user applies force to the handle which creates an applied pressure on the body ranging between 0.01 N/mm² and 1 N/mm². In some embodiments, the optimal range of pressures during the testing is 0 N/mm² to 0.5 N/mm². The probe device may be used to dynamically assess vasculature at a target site or area of interest. Typically, the probe device is held against the skin of the subject for a period of time ranging between 1 s and 300 s. In some embodiments, the preferred testing protocol comprises continuous measurements at the following intervals: (1) 60 seconds at 0 N/mm² of applied pressure (“baseline”); (2) 30 seconds at 0.1 N/mm² of applied pressure; (3) 30 seconds at 0.25 N/mm² of applied pressure; (4) 30 seconds at 0.35 N/mm² of applied pressure; (5) 30 seconds at 0.5 N/mm² of applied pressure; and (6) 30 seconds at baseline (0 N/mm² of applied pressure). In some embodiments, a preferred testing protocol involves continuous measurements at the following intervals: (1) 60 seconds at baseline (0 N/mm² applied pressure); (2) 30 seconds at 0.1 N/mm² of applied pressure; (3) 30 seconds at baseline; (4) 30 seconds at 0.25 N/mm² of applied pressure′ (5) 30 seconds at baseline; (6) 30 seconds at 0.35 N/mm² of applied pressure; (7) 30 seconds at baseline; (8) 30 seconds at 0.5 N/mm² of applied pressure; and (9) 30 seconds at baseline. Based on these protocols, we can derive the relationship between the change in total hemoglobin (HbT) concentration as a function of changes in applied pressure, the time it takes for HbT to plateau at each pressure application, the change in oxygen saturation as a result of applied pressure, and the time it takes for HbT to return to baseline after each pressure application (for protocol 2 only). These values are expected to differ between healthy subjects and patients with PAD, and among PAD patients with varying degrees of severity of the disease. In some embodiments, the probe device allows for one or more cutoff threshold values for each of these metrics which can be used to diagnose the presence of PAD, the severity of PAD, and the likelihood of a patient to respond to a surgical intervention. In some embodiments, the disclosed devices can be used for the measurement of the vascular territories in the legs and feet called angiosomes. Angiosomes cover distinct three-dimensional blocks of tissue, each of which is fed by one or more distinct source arteries such as the medial plantar artery, lateral plantar artery, posterior tibial artery (PTA), anterior tibial artery (ATA), and peroneal artery (PA). In PAD patients, vessels within certain angiosomes can be affected to a larger degree than others, depending on the location of the blockage the patient is suffering from. For example, while a blockage in the femoral artery may affect perfusion in the entire foot, a blockage in the anterior tibial artery will affect perfusion in the arcuate artery more so than the posterior tibial artery would. Various embodiments of the present invention can take advantage of the angiosome configuration of the lower legs and feet to help identify the origin of various problems (e.g., ulcerations, claudication, etc.).

The disclosed probe devices generally comprise one or more computing systems comprising a processor and a non-transitory computer-readable medium with instructions stored thereon, which when executed by a processor, performs at least some of the steps of any disclosed method.

### Computing Device

In some embodiments, devices 100, 200 may further comprise any number of computers electronically and communicatively connected to the device (e.g., light sources, photodetectors, force sensors). In some embodiments, the computer may comprise one or more computer 400 as contemplated herein.

In some aspects of the present invention, software executing the instructions provided herein may be stored on a non-transitory computer-readable medium, wherein the software performs some or all the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written, compiled, or interpreted, in any programming language known in the art, including but not limited to C, C++, C #, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller (e.g., microcontroller 180), a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 3B and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 3B depicts an illustrative computer architecture for a computer 400 for practicing the various embodiments of the invention. The computer architecture shown in FIG. 3B illustrates a conventional personal computer, including a central processing unit 450 (“CPU”), a system memory 405, including a random access memory 410 (“RAM”) and a read-only memory (“ROM”) 415, and a system bus 435 that couples the system memory 405 to the CPU 450. A basic input/output system containing the basic routines that help to transfer information between elements within the computer, such as during startup, is stored in the ROM 415. The computer 400 further includes a storage device 420 for storing an operating system 425, application/program 430, and data.

The storage device 420 is connected to the CPU 450 through a storage controller (not shown) connected to the bus 435. The storage device 420 and its associated computer-readable media provide non-volatile storage for the computer 400. Although the description of computer-readable media contained herein refers to a storage device, such as a hard disk or CD-ROM drive, it should be appreciated by those skilled in the art that computer-readable media can be any available media that can be accessed by the computer 400.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 400 may operate in a networked environment using logical connections to remote computers through a network 440, such as TCP/IP network such as the Internet or an intranet. The computer 400 may connect to the network 440 through a network interface unit 445 connected to the bus 435. It should be appreciated that the network interface unit 445 may also be utilized to connect to other types of networks and remote computer systems.

The computer 400 may also include an input/output controller 455 for receiving and processing input from a number of input/output devices 460, including a keyboard, a mouse, a touchscreen, a camera, a microphone, a controller, a joystick, or other type of input device. Similarly, the input/output controller 455 may provide output to a display screen, a printer, a speaker, or other type of output device. The computer 400 can connect to the input/output device 460 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 420 and/or RAM 410 of the computer 400, including an operating system 425 suitable for controlling the operation of a networked computer. The storage device 420 and RAM 410 may also store one or more applications/programs 430. In particular, the storage device 420 and RAM 410 may store an application/program 430 for providing a variety of functionalities to a user. For instance, the application/program 430 may comprise many types of programs such as a word processing application, a spreadsheet application, a desktop publishing application, a database application, a gaming application, internet browsing application, electronic mail application, messaging application, and the like. According to an embodiment of the present invention, the application/program 430 comprises a multiple functionality software application for providing word processing functionality, slide presentation functionality, spreadsheet functionality, database functionality and the like.

The computer 400 in some embodiments can include a variety of sensors 465 for monitoring the environment surrounding and the environment internal to the computer 400. These sensors 465 can include a Global Positioning System (GPS) sensor, a photosensitive sensor, a gyroscope, a magnetometer, thermometer, a proximity sensor, an accelerometer, an inertial measurement unit (IMU), a transducer, a microphone, biometric sensor, barometer, humidity sensor, radiation sensor, or any other suitable sensor.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Pressure Probe for Assessment of Vascular Health

The disclosed innovation is a medical device that uses pressure-dependent optical transmission data to assess the health of a vascular bed as found throughout the body. One example is peripheral arterial disease (PAD). PAD, which affects approximately 8 to 12 million people in the USA, is a condition in which the arteries that carry blood to the legs, arms, and other parts of the body become narrowed or blocked, reducing blood flow to the affected area. This can cause symptoms such as pain in the legs or feet, cramping, and difficulty walking. PAD is usually caused by the buildup of plaque in the arteries, a condition known as atherosclerosis. Risk factors for PAD include smoking, high blood pressure, high cholesterol, and diabetes. PAD can be diagnosed with a physical exam, blood tests, and imaging tests. Treatment options include lifestyle changes, medication, and procedures such as angioplasty or bypass surgery.

Over the last 5 years, measurement systems were developed that use light to probe the vascular health in the feet of PAD patients. These systems use patches that contain laser diodes that emit light at multiple wavelengths and photodetectors that measure light intensity. These patches are placed on different locations on the lower legs and feet. Once placed, the laser diodes inject light into the surrounding tissue and the photodetectors measure how much light comes out of the surrounding tissue. When the light propagates from the laser diode through the tissue to the photodetectors it interacts with the blood in the tissue. For example, more blood in the area under the patch causes more light to be absorbed, and the intensity of light measured by the photodetectors is reduced. Measuring the light absorption at multiple wavelengths provides information about the amount of blood as well as the amount of oxygen and other chromophores in the lower legs and feet.

Employing these systems in studies with PAD patients and healthy volunteers revealed a correlation between parameters derived from optical data and vascular health [A. Marone, J. W. Hoi, C. J. Fong, Y. Kim, H. K. Kim, D. R. Bajakian, and A. H. Hielscher, “Using dynamic vascular optical spectroscopy to evaluate peripheral arterial disease (PAD) in patients who undergo a vascular intervention,” in Optical Tomography and Spectroscopy of Tissue XIII, (SPIE 2019), pp. 108740E; A. Marone, J. W. Hoi, M. Khalil, H. K. Kim, G. Shrikhande, R. Dayal, D. Bajakian, and A. H. Hielscher, “Modeling of the hemodynamics in the feet of patients with peripheral artery disease”. Biomedical Optics Express. 10(657), (2019); N. Maheshwari, A. Marone, M. Altoć, H. K. Kim, D. R. Bajakian, A. H. Hielscher, “Pilot study on monitoring ulcer healing with diffuse optical imaging in a patient cohort affected by peripheral arterial disease (PAD),” in Optical Diagnostics and Sensing XXII: Toward Point-of-Care Diagnostics, (SPIE 2022), pp. 119680A; Marone, A., Maheshwari, N., Kim, S. H., Bajakian, D. R., & Hielscher, A. (2022). Dynamic vascular optical spectroscopy for monitoring peripheral arterial disease patients undergoing a surgical intervention. Frontiers in Photonics]. Additionally, a classification potential was shown (86% sensitivity, 100% specificity) between wound-healing outcome for PAD patients after a surgical intervention and optically derived parameters from these systems [N. Maheshwari, A. Marone, M. Altoć, H. K. Kim, D. R. Bajakian, A. H. Hielscher, “Pilot study on monitoring ulcer healing with diffuse optical imaging in a patient cohort affected by peripheral arterial disease (PAD),” in Optical Diagnostics and Sensing XXII: Toward Point-of-Care Diagnostics, (SPIE 2022), pp. 119680A].

The disclosed device adds pressure information to the optical data. Optical absorption measurements are taken as before; however, these measurements were performed under different applied loads. This results in data sets that show light absorption as a function of the pressure with which the patch or probe is pressed against the skin. This additional information should provide better metrics for vascular health and may assist physicians in diagnosing and monitoring PAD or other vascular diseases.

A prototype of such a system was developed and was used in making measurements on healthy volunteers. The prototype (see FIGS. 2A-2F) comprises the following components. In some embodiments, the hardware is comprised of a two-part pressure probe 3D printed with ABS. In some embodiments, part one is a probe cap comprising one hollow cylindrical tube (with wall width of 2.5 mm) and encases a smaller solid cylindrical tube or body. In some embodiments, the user grips the larger cylinder (probe cap or handle) and applies a downward force, wherein the force is also applied to smaller cylinder body (two are attached at the top, forming the top of the probe cap). In some embodiments, the small cylinder has an 8 mm diameter.

In some embodiments, part two is a probe body and comprises a cylindrical tube with 20 mm diameter. In some embodiments, the top half of probe body is hollow, where the small cylinder from part one (probe cap) is inserted. In some embodiments, after insertion, the probe cap applies pressure to solid portion of probe body (bottom half of probe body), wherein this causes the probe body to exert force on tissue. In some embodiments, the top half of probe body fits between the two cylindrical tubes that comprise the probe cap. In some embodiments, a 5.75 mm width ring on the bottom face of probe body improves attachment to tissue and adherence of sources and detector to tissue. In some embodiments, a Tegaderm film is placed over top of ring and surrounding tissue and adheres to both and holds probe body to tissue. In some embodiments, the bottom face of probe has two laser diodes and one photodetector flush with the face of the probe body that make contact with the tissue. In some embodiments, a solid portion of probe body (bottom half) has two slots for source and detector connector casings. In some embodiments, two detachable snap-in cases for source and detector connectors are also 3D printed with ABS. In some embodiments, the snap-in cases attach to bottom half of probe body via slots.

In some embodiments, the system comprises two laser diodes with wavelengths 780 nm and 850 nm, where each is around 10 mm away from one silicon photodetector and all are on bottom face of probe body. In some embodiments, the system has a tissue probing depth of ˜5 mm. In some embodiments, the laser diodes have 5.6 mm diameter, 5-10 mW power, and wavelength is modulated in amplitude between 0V and 3.3V at 5 kHz. In some embodiments, the laser diodes are from Thor Labs: L780P010 and L850P010. In some embodiments, the photodetector is from Hamamatsu: S1337-33BR. In some embodiments, one force sensor is inserted into probe body via a slit in the bottom half of the probe body. In some embodiments, the force sensor is from SingleTact: 8 mm Diameter Sensor, 100N Force. In some embodiments, a portion of the probe cap that exerts force on probe body (small cylinder of probe cap) contacts force sensor and pushes it against the bottom of the slit. In some embodiments, the force sensor continuously records applied force and sends information to a graphical user interface (GUI) to be displayed. In some embodiments, the data from source-detector pairs are acquired by the probe hardware at ˜10.24 Hz. In some embodiments, the data from force sensor are acquired by SingleTact control board at ˜10.24 Hz.

In some embodiments, the data from source-detector pairs and force sensor are displayed in real-time on a GUI. In some embodiments the GUI is can be produced in MATLAB. In some embodiments, users can start and stop data acquisition through the MATLAB GUI. In some embodiments, the GUI displays both the signals from source-detector pairs and force sensor readings on two adjacent graphs in real-time. In some embodiments, the applied force is also reported numerically in real-time on the GUI. In some embodiments, the users can store and analyze data using the MATLAB GUI, including reconstructing data to get the oxygenated, deoxygenated, and total hemoglobin concentrations, oxygen saturation, and information on pulse amplitude versus applied pressure. Although a MATLAB GUI is described, it should be appreciated that a device specific GUI for numerous operating platforms may also be designed.

Current diagnostic and monitoring methods for peripheral arterial disease (PAD) have a relatively low sensitivity or are unable to characterize perfusion in the feet (which is a common location for blocked arteries/manifestation of the disease). Nearly 30% of PAD patients who undergo surgery require a second surgical intervention within 12 months due to persistence of symptoms. There is currently no way to predict how a patient will respond to surgical intervention prior to the surgery. For patients that need multiple surgeries (are in the 30%), a different initial treatment may be more effective-knowing this in advance is key. A diagnostic and monitoring device with higher sensitivity would also be helpful due to current limitations with using the available existing technology to assist diabetic patients or to characterize arteries in the feet.

The disclosed pressure probe is the first probe to measure the effects of applied force on blood perfusion at the point of application (the pressure application and absorption information are localized to the same tissue area). The disclosed probe applies pressure at the same location as the source-detector pairs which acquire blood perfusion information. Local application of pressure and response to pressure may provide more sensitive information about vascular health. The disclosed pressure probe has a probing depth of 5 mm into the tissue, which reaches beyond the superficial tissue layers. The probe is small and easy to handle (32 mm diameter at point of contact). With the disclosed device, it is possible to get blood perfusion information for arteries in the lower extremities and feet. Accurate information on blood flow in the feet has been hard to acquire with current technologies.

### Example 2: Design of Handheld Probe to Monitor the Relationship Between Applied Pressure and Blood Volume Changes

A handheld probe combining pressure sensing with dynamic vascular near-infrared spectroscopy was designed to observe the relationship between applied pressure and change in hemoglobin concentration, which may be related to vascular health.

Peripheral arterial disease (PAD), a vascular disease caused by a buildup of plaque in the arteries, currently effects 8 to 12 million people in the United States alone [H. Johnston-Cox, D. Kadian-Dodov, and J. W. Olin, “Diagnosis and management of diseases of the peripheral arteries” in Fuster and Hurst's The Heart, 15th ed., Chapter 26, V. Fuster, et al. Eds. McGraw Hill (2022)]. In previous research, a correlation was found between parameters derived from optical data and vascular health [A. Marone, J. W. Hoi, C. J. Fong, Y. Kim, H. K. Kim, D. R. Bajakian, and A. H. Hielscher, “Using dynamic vascular optical spectroscopy to evaluate peripheral arterial disease (PAD) in patients who undergo a vascular intervention,” in Optical Tomography and Spectroscopy of Tissue XIII, (SPIE 2019), pp. 108740E; A. Marone, J. W. Hoi, M. Khalil, H. K. Kim, G. Shrikhande, R. Dayal, D. Bajakian, and A. H. Hielscher, “Modeling of the hemodynamics in the feet of patients with peripheral artery disease”. Biomedical Optics Express. 10(657), (2019); N. Maheshwari, A. Marone, M. Altoć, H. K. Kim, D. R. Bajakian, A. H. Hielscher, “Pilot study on monitoring ulcer healing with diffuse optical imaging in a patient cohort affected by peripheral arterial disease (PAD),” in Optical Diagnostics and Sensing XXII: Toward Point-of-Care Diagnostics, (SPIE 2022), pp. 119680A; Marone, A., Maheshwari, N., Kim, S. H., Bajakian, D. R., & Hielscher, A. (2022). Dynamic vascular optical spectroscopy for monitoring peripheral arterial disease patients undergoing a surgical intervention. Frontiers in Photonics]. Additionally, a classification potential was shown (86-89% sensitivity, 100% specificity) between wound outcome for PAD patients after a surgical intervention and optically derived parameters [N. Maheshwari, A. Marone, M. Altoć, H. K. Kim, D. R. Bajakian, A. H. Hielscher, “Pilot study on monitoring ulcer healing with diffuse optical imaging in a patient cohort affected by peripheral arterial disease (PAD),” in Optical Diagnostics and Sensing XXII: Toward Point-of-Care Diagnostics, (SPIE 2022), pp. 119680A; Maheshwari, N., Marone, A., Altoć, M., Kim, S. H., Bajakian, D. R. and Hielscher, A. H., 2022. Postintervention monitoring of peripheral arterial disease wound healing using dynamic vascular optical spectroscopy. Journal of biomedical optics, 27(12), p. 125002]. However, this classification was dependent on data acquired 3-4 weeks after a surgical revascularization procedure. The addition of pressure information and the hemodynamic response to pressure may provide better metrics for vascular health and may assist physicians in assessing PAD patient response to surgery prior to the intervention.

The handheld pressure probe included two 5.6 mm diameter laser diodes with 10 mW power at 780 nm and 850 nm (L780P010, L850P010 Thor Labs), one silicon photodetector (S1337-33BR, Hamamatsu), and one 8 mm diameter 100N force sensor (SingleTact). The diodes are approximately 10 mm from the photodetector (FIGS. 2A-2F) and each wavelength is modulated in amplitude between 0V and 3.3V at a frequency of 5 kHz. A ring at the base of the probe body is taped to the skin using Tegaderm, which ensures adhesion between the probe and tissue throughout the acquisition process.

A force is manually applied to the probe by gripping the probe cap and pressing down. The cap internally connects with the body of the probe and pushes the body of the probe into the tissue above an artery of interest (FIGS. 2A-2F). The force exerted on the probe is read by the force sensor and displayed as pressure [N/mm²] in real-time using a MATLAB GUI.

Optical data from the two source-detector pairs are simultaneously acquired by the probe hardware and displayed in real-time on the MATLAB GUI. The acquisition rate for both the optical and pressure data is 10.24 Hz.

A protocol was developed for use on three healthy volunteers to validate the handheld probe's ability to monitor the relationship between pressure and hemodynamics. The acquisition process included three main components: an initial 30 second baseline measurement with no applied force, four 20 second stages with a constant applied force that increased at each stage, and a final 30 second baseline measurement after the applied force was released. Large changes in applied force were avoided during each 20 second loading stage using feedback from the real-time plot of the force sensor reading which ensured the force was adjusted appropriately to ensure a relatively constant load. The acquired optical data was processed using a previously developed diffusion-theory-based PDE-constrained multispectral reconstruction algorithm [A. Marone et al., Biomedical Optics Express. 10 (657), (2019)]. From this, the total hemoglobin concentration (HbT) over time was determined.

Validation tests were run at three different locations in the lower extremities: the medial plantar artery, the dorsalis pedis artery, and the posterior tibial artery. There were five changes in applied load at which hemodynamics were observed: P01—change from initial/baseline force to first application of force; P12—change from first to second application of force; P23—change from second to third application of force; P34—change from third to fourth application of force; P4E—change from fourth application of load to end/baseline force.

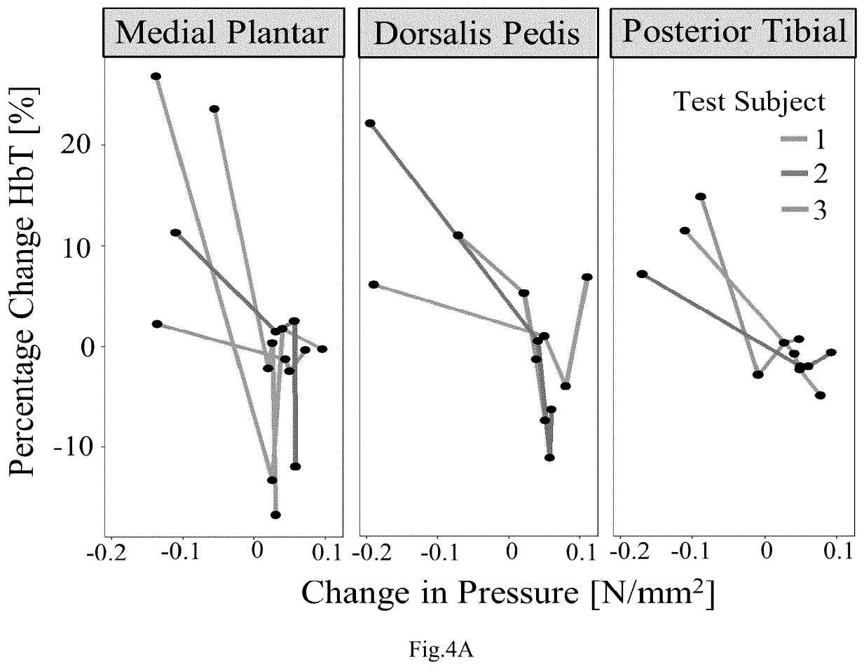

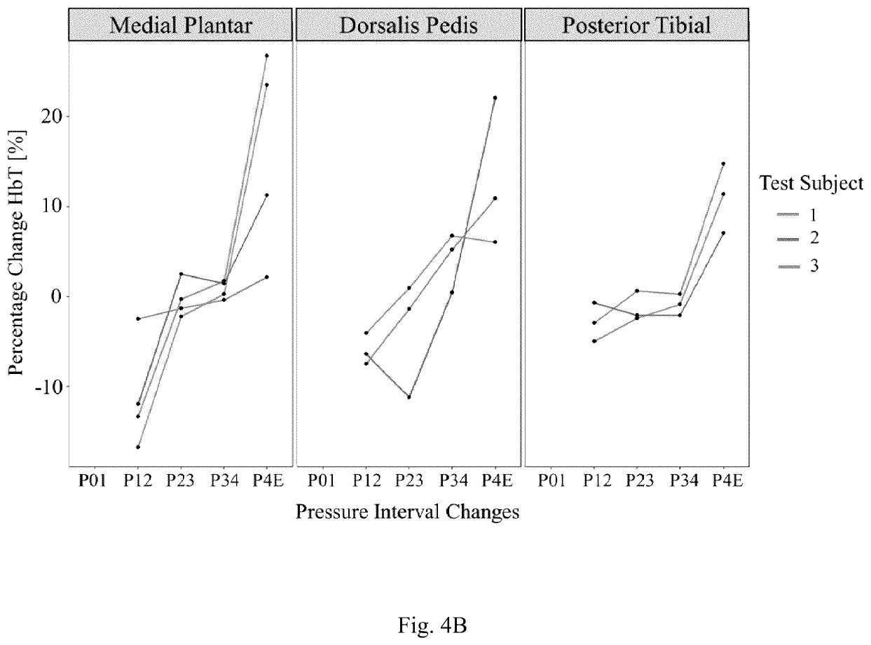

Results from the experiment show that there were changes in total hemoglobin concentration in response to applied force at each location and these responses seem to follow similar trends across subjects at each location (FIGS. 4A-4B).

A Spearman's rank-order correlation analysis determined that there was a strong correlation (R=−0.77, p<0.05) between location and the percent change in total hemoglobin concentration at P34. Spearman's rank-order analysis also found a strong correlation (R=−0.7, p<0.05) between the P12 and P4E metrics.

These preclinical results indicate that it is possible to simultaneously collect applied pressure data and optical data from different locations on the lower extremities. The observed correlation between P12 and P4E matches the expected, as it was expected for the first application of force to have the largest effect HbT, and it was expected for the baseline HbT to be similar at the beginning and end of the protocol. Additionally, the strong correlation between P34 and location indicates that the relationship between pressure and hemodynamics may be useful in monitoring regional vasculature.

### Example 3: Preliminary Study Monitoring the Relationship Between Applied Pressure and Blood Volume for the Assessment of Vascular Health

Peripheral arterial disease (PAD) affects an estimated 8.5 million people in the United States. Diagnostic tools to identify PAD continue to have low sensitivity for patients with poor vascular health in the lower extremities. A novel handheld probe device as disclosed herein may address these limitations. The probe combines dynamic vascular optical spectroscopy with pressure sensing to monitor the relationship between applied force and blood volume changes in tissues of interest. The applied pressure at which the measured pulse amplitude goes to zero is expected to differ between healthy and PAD subjects. The probe has a 20 mm diameter, with the bottom face housing two infrared sources (wavelength λ=780 nm and 850 nm) and one silicon photodiode located around 10 mm apart. The probe continuously records blood perfusion data based on the absorption of light by the tissue under the probe at a frame rate of 10.24 frames per second. There is also a force sensor that continuously records load applied to the tissue under the probe. During data acquisition, the applied load is gradually increased, resulting in dynamic changes of the monitored light intensity signals. The pressure necessary for the pulse amplitude to reach zero can be extracted as can the relationship between applied pressure and pulse amplitude from the recorded signal traces. Here, the progress of a first preclinical study monitoring multiple vascular locations from 5 healthy volunteers is reported. Preliminary results show that on average there is a 2.7% change in signal per 7.4 N change in applied force.

Peripheral arterial disease (PAD) affects at least 8.5 million people in the United States alone [H. Johnston-Cox, D. et al., in Fuster and Hurst's The Heart, 15th ed., Chapter 26, V. Fuster, et al. Eds. McGraw Hill (2022)]. The disease is caused by a buildup of plaque in the arteries and predominantly affects the lower extremities. Early symptoms include discomfort or pain in the legs and feet. If left untreated, patients may experience more severe symptoms including gangrene, non-healing wounds, and amputation [A. Stoyioglou and M. R. Jaff, J. Vasc. Interv. Radiol. 15(11), 1197-1207 (2004); H. L. Gornik and J. A. Beckman, Circulation 111(13), e169-e172 (2005); R. A. G. Patel et al., Curr. Probl. Cardiol. 45(7), 100402-100429 (2020); J. D. Santilli and S. M. Santilli, Am. Fam. Physician. 59(7), 1899-1908 (1999)]. Physicians will commonly recommend endovascular revascularization surgery to treat PAD. Common surgical interventions target plaque filled arteries by using an inflatable balloon attached to a catheter to compress plaque against the artery wall and restore blood flow to the artery. Unfortunately, 30% of patients require a second intervention within 12 months after the initial surgery due to persistence of symptoms [J. A. Beckman et al., Circulation Research, 128(12), 318261 (2021); J. H. Rogers and J. R. Laird., Circulation. 116(18), 715433 (2007); B. H. Gray, et al., J Vasc Surgery. 25(1), 74-83 (1997)] Current monitoring methods such as the ankle-brachial index (ABI) and arterial duplex ultrasound (A-DUS) do not provide information on distal perfusion in the foot and are unable to predict a patient's response to surgery prior to the intervention. Additionally, ABI lacks sensitivity for PAD patients with diabetes, who make up 20-40% of the patient population [J. D. Santilli and S. M. Santilli, Am. Fam. Physician. 59(7), 1899-1908 (1999); T. Yoshimura, E. et al., Diabetes Care 29(8), 1884-1890 (2006); D. Xu et al., Vasc. Med. 15(5), 361-369 (2010)].

In previous research, a correlation between parameters derived from optical data and vascular health were derived using a dynamic vascular optical spectroscopy system (DVOS) [A. Marone et al., in Optical Tomography and Spectroscopy of Tissue XIII, (SPIE 2019), pp. 108740E; A. Marone et al., Biomedical Optics Express. 10(657), (2019); N. Maheshwari et al., Optical Diagnostics and Sensing XXII: Toward Point-of-Care Diagnostics, (SPIE 2022), pp. 119680A; Maheshwari, N. et al., Journal of biomedical optics, 27(12), p. 125002; Marone, A. et al., Frontiers in Photonics 3 (2022): 938144]. It was also shown that DVOS has the potential to classify patient outcomes shortly after a surgical intervention [N. Maheshwari et al., Optical Diagnostics and Sensing XXII: Toward Point-of-Care Diagnostics, (SPIE 2022), pp. 119680A]. Developed was a handheld probe based on the DVOS technology which adds hemodynamic response to force application as another measurement for determining vascular health as a potential way to determine patient response to intervention prior to a revascularization surgery. As described herein, data is introduced from this handheld pressure probe at multiple vascular locations from 5 healthy volunteers. A change in signal is expected with the application of force and characterizing the pressure at which the pulse amplitude signal is zero is of interest.

The handheld probe included two laser diodes with wavelengths of 780 nm and 850 nm (L780P010, L850P010 Thor Labs). These sources are flush with the bottom face of the probe, which is 20 mm in diameter. One silicone photodetector (S1337-33BR, Hamamatsu) is located approximately 10 mm away from either source and is also flush with the probe face. The wavelengths have 5-10 mW power and are modulated in amplitude between 0V and 3.3V at a frequency of 5 kHz. Optical data from the source-detector pairs are acquired at a rate of 10.24 Hz and displayed on a MATLAB GUI.

A force is manually applied to the probe, and this force is continuously recorded by a force sensor at 10.24 Hz and displayed on the MATLAB GUI in N/mm².

A protocol was developed to test the probe's ability to monitor the relationship between hemodynamics and pressure on 5 healthy patients. The acquisition process had an initial 30 second baseline measurement with no applied force, four consecutive 20 second stages with a constant force that increased at each stage, and a final 30 second baseline measurement after the force was released. At each force stage, the user regulated the application of force via feedback from the force sensor to apply the same amount of force during each 20 second interval. The acquired optical data was processed using a previously developed diffusion-theory-based PDE-constrained multispectral reconstruction algorithm which allows us to determine the total hemoglobin (HbT) over time [A. Marone et al., Biomedical Optics Express. 10(657), (2019)]. Validation tests were run at multiple locations on the forearms and upper arm.

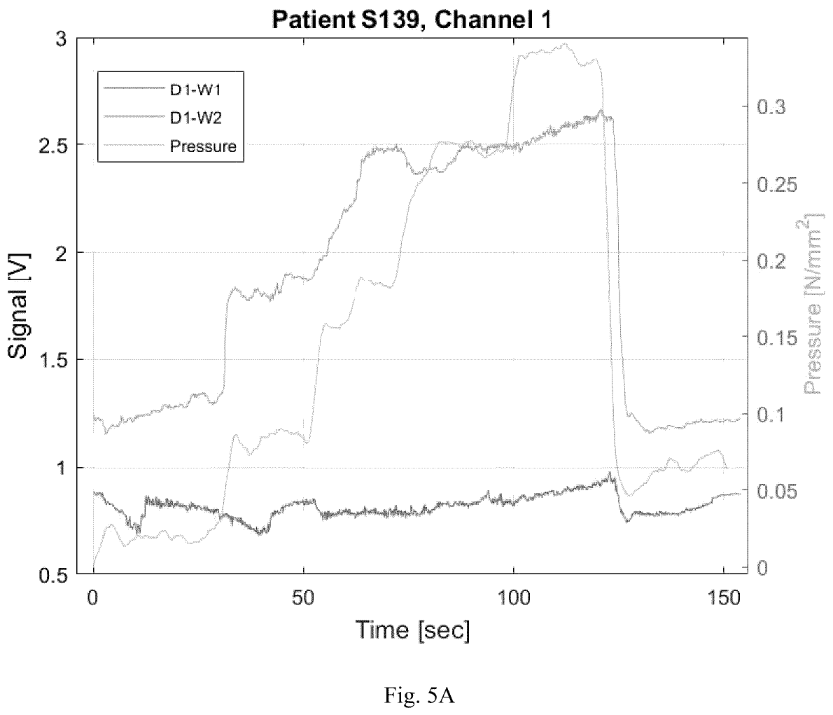

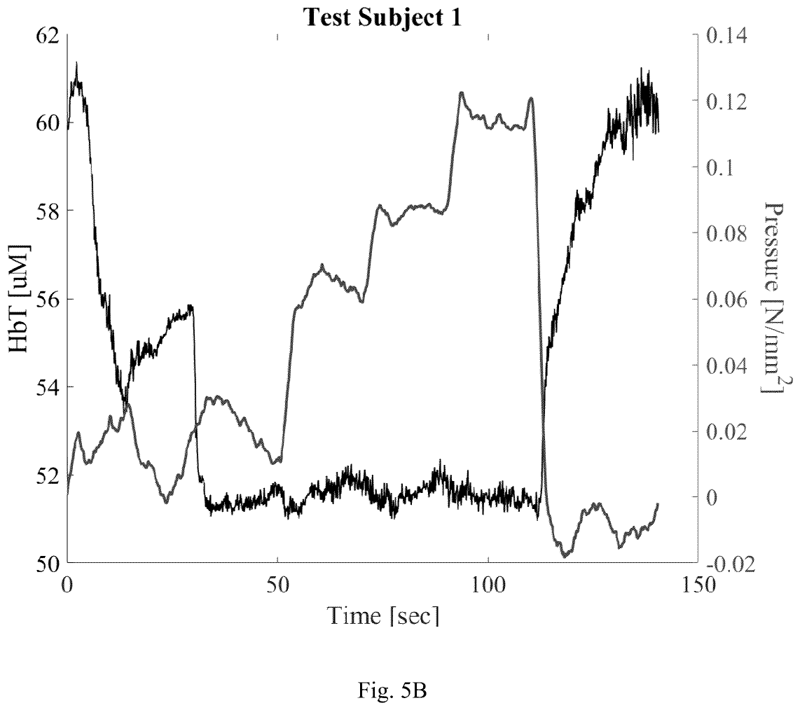

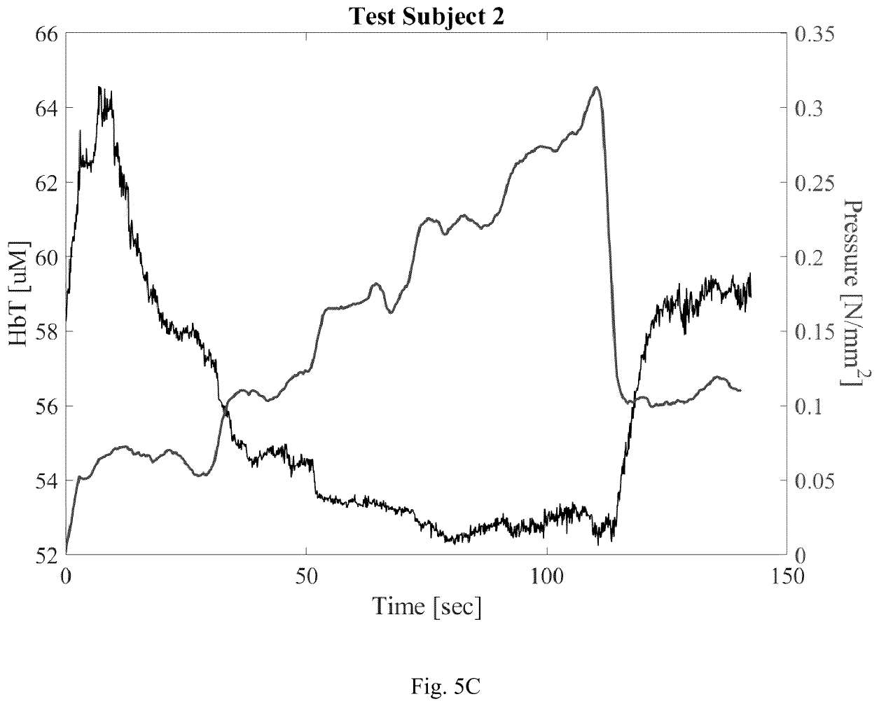

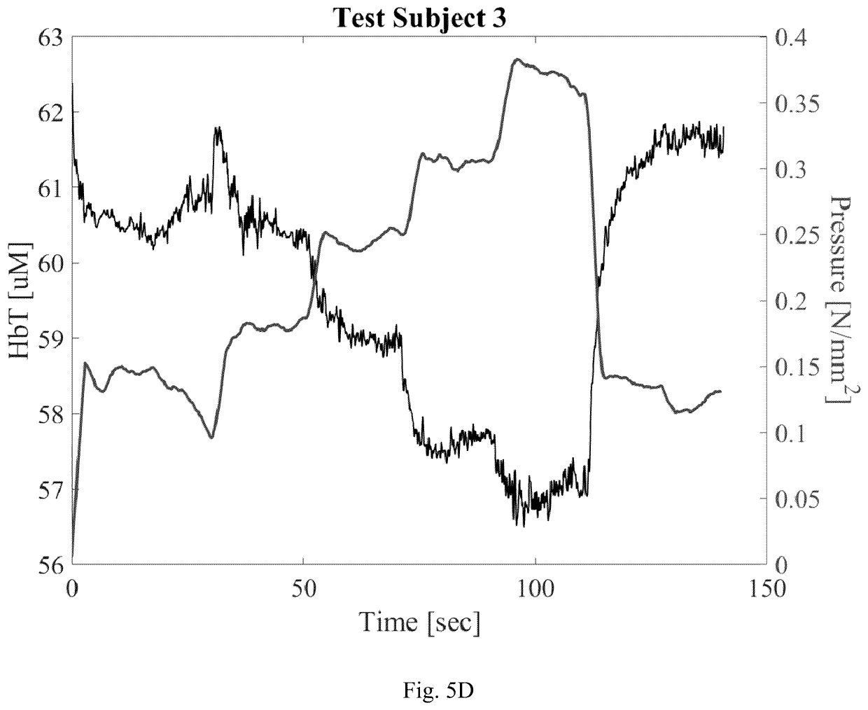

Results of this experiment show that there was an observed increase in signal relative to an increase in applied pressure (see FIG. 5A) and decrease in HbT relative to an increase in applied pressure (see FIG. 5B) during the probe acquisition procedure. This pattern was observed for all patient tests.

On average, there was a 2.7% change in signal per 7.4 N change in force. This increase in signal was observed for both the 780 nm and 850 nm wavelengths. Upon release of force, the signal (and therefore HbT) values returned to baseline (FIG. 5A-5D). Interestingly, there was a difference in HbT response to applied force in Test Subjects 1 and 2 (both of whom had a history of smoking) as compared to Test Subject 3 (with no history of smoking; FIG. 5D). In the subjects with a history of smoking, the initial application of pressure caused a significantly greater decrease in HbT than the subsequent increases in applied force. In the subject with no smoking history, each new application of force had a similar effect on HbT (FIGS. 5C-5D).

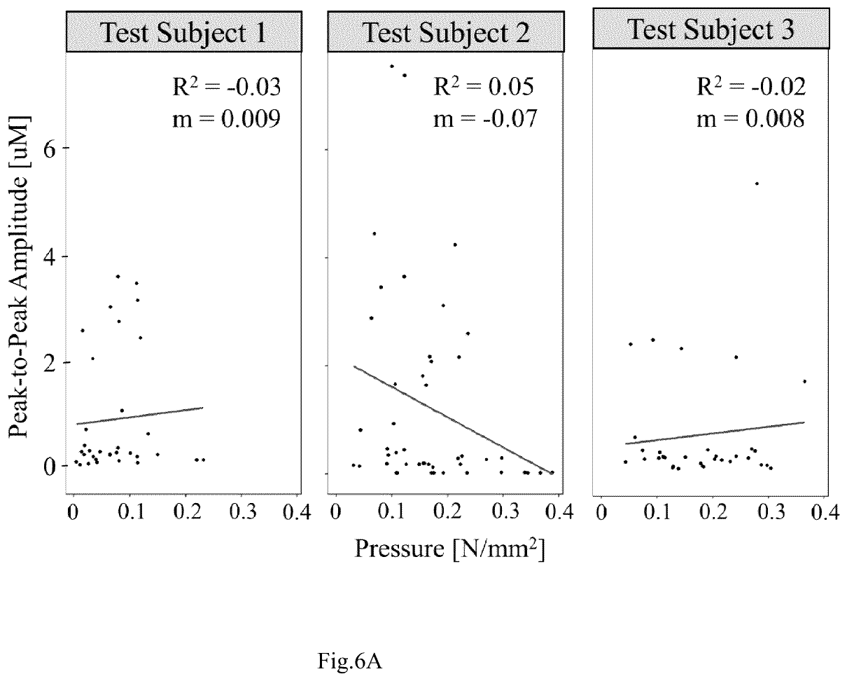

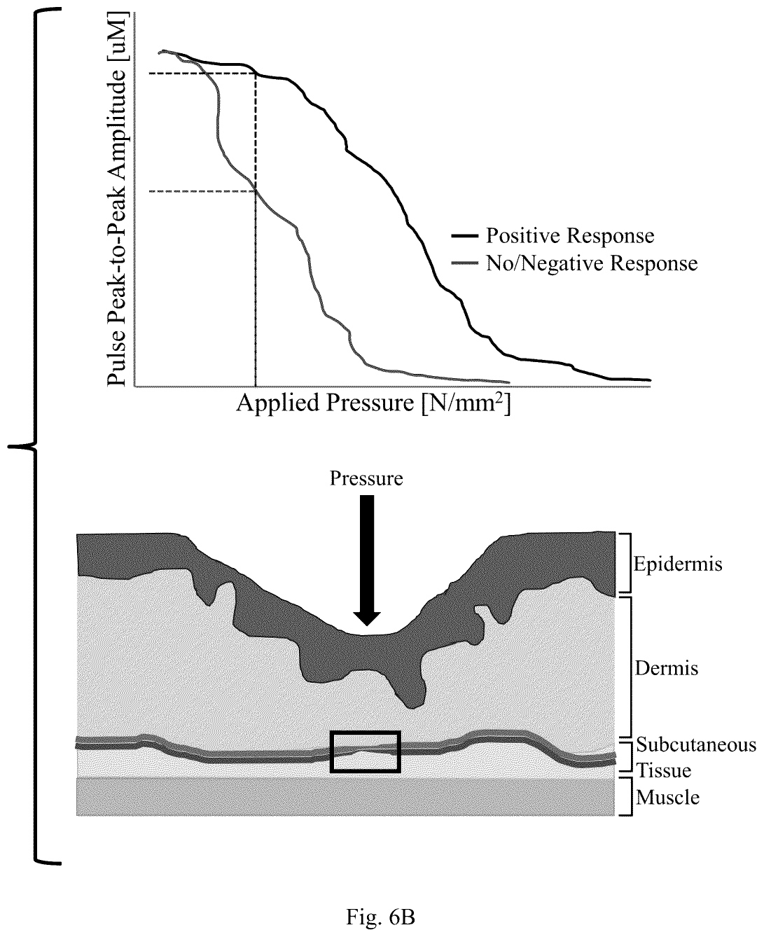

The change in pulse amplitude in response to applied force was volunteer dependent. The expected decrease in pulse amplitude was not observed with respect to an increase in force for all tests (FIG. 6A). For one volunteer, this pattern was observed (FIG. 6A) but was not significant, while for the other volunteers, there was a seemingly random change in pulse amplitude relative to change in force.

A change in signal relative to applied force was observed for all 5 of the healthy volunteers that data were acquired from. On average the signal changed 2.7% per 7.4 N of force applied. These results confirm that there is a hemodynamic response to pressure at multiple distinct vascular locations. An unexpected relationship between the pulse peak to peak amplitude and the applied force was observed, which may indicate that pulse amplitude is not a reliable measure of vascular health. The disclosed data acquisition protocol can be used with PAD patients to see if the response to pressure differs between healthy and PAD patients. In some embodiments, the shape of the HbT curve is characterized in response to applied pressure. In some embodiments, HbT behavior in PAD patients is compared and contrasted with the responses of healthy subjects. In some embodiments, the pulse amplitude response to pressure may be more consistent in PAD patients, in which case the pulse peak-to-peak amplitude may also be investigated. The combination of optical data and applied pressure provides valuable information on vascular health that leads to better diagnostics and treatment for PAD patients.

### Example 4: Preliminary Study to Assess Vascular Health for Future Use in Patients with Peripheral Arterial Disease Using Pressure Dependent Dynamic Optical Spectroscopy

Peripheral arterial disease (PAD) affects over 8.5 million people in the United States. Diagnostic tools to identify PAD continue to have low sensitivity for patients with diabetes and/or with poor vascular health in the small vessels of the lower extremities. A handheld device disclosed herein may address these limitations. The device combines dynamic vascular optical spectroscopy (DVOS) with pressure sensing to monitor the relationship between applied pressure and blood volume changes in tissues of interest, which is expected to differ between healthy and PAD subjects. The disclosed probe is 20 mm in diameter with the bottom face housing two infrared sources (wavelength λ=780 nm and 850 nm) and one silicon photodetector located around 10 mm from each source. The DVOS system continuously records the reflected light intensity from the local tissue at a frame rate of 10.24 frames per second. Simultaneously, the load applied by the probe to the tissue surface is measured continuously with a force sensor at the same frame rate. During data acquisition, the applied load is gradually increased, resulting in dynamic changes of the monitored DVOS signals. These recorded signals provide information on the response of the local tissue perfusion to changes in applied pressure. Disclosed in this example is a preclinical study monitoring 3 vascular locations in the lower extremities of 3 healthy volunteers. Preliminary results suggest that on average there is a 150% change in total hemoglobin concentration per 1 N/mm² change in applied pressure. It was expected that these changes would be significantly smaller in PAD patients.

Peripheral arterial disease (PAD) affects at least 8.5 million people in the United States alone. [H. Johnston-Cox, D. et al., in Fuster and Hurst's The Heart, 15th ed., Chapter 26, V. Fuster, et al. Eds. McGraw Hill (2022)] The disease is caused by a buildup of plaque in the arteries and predominantly affects the lower extremities. Early symptoms include discomfort or pain in the legs and feet. If left untreated, patients may experience more severe symptoms including gangrene and non-healing wounds. [A. Stoyioglou and M. R. Jaff, J. Vasc. Interv. Radiol. 15(11), 1197-1207 (2004); H. L. Gornik and J. A. Beckman, Circulation 111(13), e169-e172 (2005); R. A. G. Patel et al., Curr. Probl. Cardiol. 45(7), 100402-100429 (2020); J. D. Santilli and S. M. Santilli, Am. Fam. Physician. 59(7), 1899-1908 (1999)] Physicians will commonly recommend endovascular revascularization surgery (such as balloon angioplasty, in which an inflatable balloon attached to a catheter compresses plaque against the artery wall to restore blood flow) to treat PAD. Unfortunately, 30% of patients require a second intervention within 12 months after their initial surgery due to persistence of symptoms. [J. A. Beckman et al., Circulation Research, 128(12), 318261 (2021); J. H. Rogers and J. R. Laird., Circulation. 116(18), 715433 (2007); B. H. Gray, et al., J Vasc Surgery. 25(1), 74-83 (1997)] Amputation is often also necessary for patients with severe PAD that do not respond to treatment. [H. Johnston-Cox, D. et al., in Fuster and Hurst's The Heart, 15th ed., Chapter 26, V. Fuster, et al. Eds. McGraw Hill (2022)] Current monitoring methods such as the ankle-brachial index (ABI) and arterial duplex ultrasound do not provide information on distal perfusion in the foot and are unable to predict a patient's response to surgery prior to the intervention. Additionally, ABI lacks sensitivity for PAD patients with diabetes, who make up 20-40% of the patient population. [J. D. Santilli and S. M. Santilli, Am. Fam. Physician. 59(7), 1899-1908 (1999); T. Yoshimura et al., Diabetes Care 29(8), 1884-1890 (2006); D. Xu et al., Vasc. Med. 15(5), 361-369 (2010)]

Common methods for diagnosing and monitoring PAD have low sensitivity in patients with diabetes because diabetics are more likely to have arterial calcifications, [Mohler III ER. Peripheral Arterial Disease: Identification and Implications. Arch. Intern. Med. 2003; 163(19): 2306-2314] and tend to have difficulty visualizing small vessels such as those in the feet. [Al-Qaisi M et al., Reports in Med. Imag. 2009; 2:25-34] Pressure dependent dynamic vascular optical spectroscopy (pDVOS) may be able to address these limitations. pDVOS may be able to differentiate between healthy subjects and PAD patients with higher accuracy than existing technology.

In previous research, a correlation was found between parameters derived from the disclosed dynamic vascular optical spectroscopy (DVOS) system and vascular health. [A. Marone et al., in Optical Tomography and Spectroscopy of Tissue XIII, (SPIE 2019), pp. 108740E; A. Marone et al., Biomedical Optics Express. 10(657), (2019); N. Maheshwari et al., Optical Diagnostics and Sensing XXII: Toward Point-of-Care Diagnostics, (SPIE 2022), pp. 119680A] It was shown that DVOS has the potential to classify patient outcomes shortly after a surgical intervention. [N. Maheshwari et al., Optical Diagnostics and Sensing XXII: Toward Point-of-Care Diagnostics, (SPIE 2022), pp. 119680A] The disclosed handheld probe adds hemodynamic response to force application as another measurement for determining vascular health. These pressure dependent DVOS (pDVOS) measurements may also be a potential way to determine a patient's response to an intervention prior to their surgery. In this example, data is introduced from the handheld pressure probe at multiple vascular locations from 3 healthy volunteers. It was expected that pDVOS signals would change with changes in applied force and further planned to characterize the hemodynamic response.

pDVOS may be able to assess which PAD patients will respond well to surgical intervention (see FIG. 6B) prior to the intervention itself. FIG. 6B is a plot displaying the expected behavior of total hemoglobin (HbT measured in uM) in response to applied pressure (N/mm²) for PAD patients who respond to treatment (positive outcome) and those who do not (negative outcome).

Methodology: The disclosed handheld probe was developed to assess the relationship between pressure and hemodynamics in the feet. The probe is comprised of a body and a cap (see FIG. 2A). While holding the probe, a force is manually applied to the cap, which causes the body to exert pressure on the tissue below the probe. A ring was added to the bottom face of the probe (see FIG. 2B) to improve its adhesion to the tissue.

The probe comprises two laser diodes with wavelengths of 780 nm and 850 nm (L780P010, L850P010 Thor Labs), one silicone photodetector (S1337-33BR, Hamamatsu) located approximately 10 mm away from these sources, and one 8 mm diameter force sensor (SingleTact). Both the sources and photodetector are located on the bottom face of the probe, which is 20 mm in diameter (see FIG. 2B). The force sensor is placed such that when the user applies pressure to the probe, the cap pushes on the force sensor. The laser diodes have 10 mW power and are modulated in amplitude between 0 V and 3.3 V at a frequency of 5 kHz. DVOS measurements from the source-detector pairs and applied force measurements from the force sensor are simultaneously and continuously acquired at a rate of 10.24 Hz and displayed on a MATLAB graphical user interface (GUI). In some embodiments, the source detector module is MAXM86161 from Analog Devices.

FIG. 2A shows a prototype probe cap (purple) and probe body (black). FIG. 2B shows an exemplary face of the probe that adheres to the tissue of interest, with two laser diodes of wavelengths 780 nm and 850 nm and a photodetector placed 10 mm from both sources.

A data acquisition protocol was developed to assess the probe's ability to monitor the relationship between hemodynamics and pressure in the small vessels of the lower extremities. This protocol was tested on 3 healthy volunteers in the medial plantar artery (foot), dorsalis pedis artery (foot), and the posterior tibial artery (calf). The acquisition process consists of an initial 30 second baseline measurement with no applied force, the addition of a constant applied force in four 20 second intervals with greater force being applied at the beginning of each interval, and a final 30 second baseline measurement after the force was released. The force applied to the probe was continuously recorded and displayed in the MATLAB GUI throughout the acquisition process. To ensure that a constant force was applied at each interval, the GUI provided the user with visual feedback on changes in applied force throughout the acquisition process. This allowed the user to regulate their application of force in response to changes measured by the force sensor. The acquired optical data was processed using a previously developed diffusion-theory-based PDE-constrained multispectral reconstruction algorithm which allowed us to determine the total hemoglobin concentration (HbT) over time. [A. Marone et al., Biomedical Optics Express. 10(657), (2019)] The HbT is reported in uM.

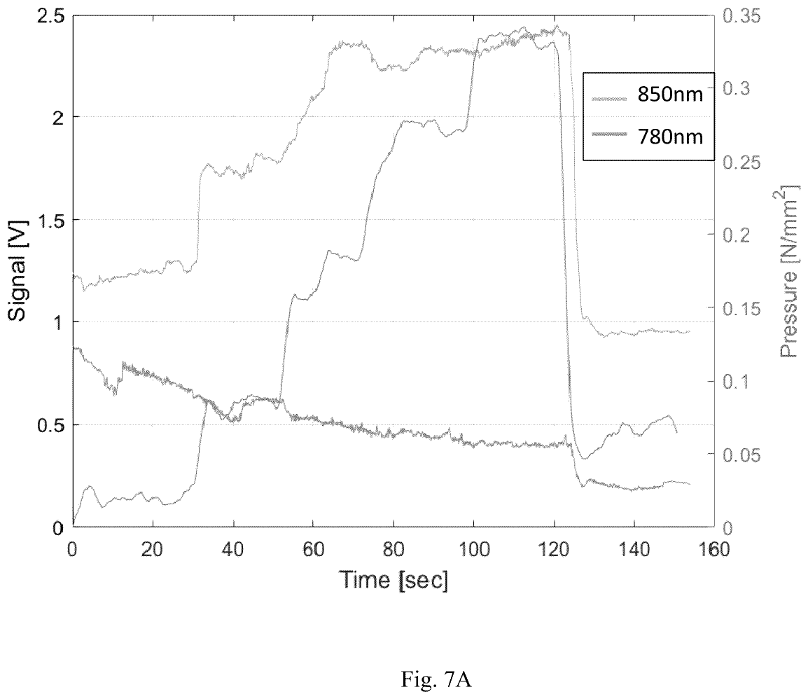

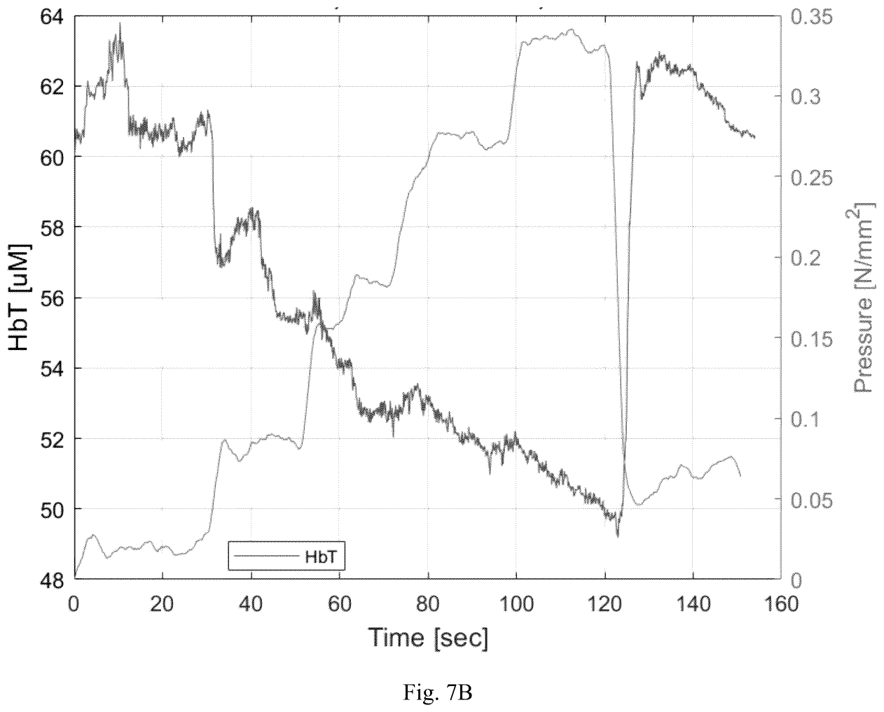

Results: There was an observed increase in signal relative to an increase in applied pressure FIG. 7A and decrease in HbT relative to an increase in applied pressure FIG. 7B during the probe acquisition procedure. The changes in HbT in response to applied force follow similar trends across participants at each measurement location (see FIGS. 4A-4B).

FIG. 7A displays Signal (V) for the 780 nm (green) and 850 nm (cyan) wavelength laser diodes and pressure (N/mm²) (orange) vs time (sec). FIG. 7B shows the corresponding HbT (uM) (blue) and pressure (N/mm²) (orange) vs time (sec) from a representative heathy volunteer test.

FIG. 4B show the percent change of total hemoglobin [%] at each change in applied force for the 3 healthy volunteers at the medial plantar (left), dorsalis pedis (center), and posterior tibial (right) angiosomes. P01—change from initial/baseline to first application of force; P12—change from first to second application of force; P23—change from second to third application of force; P34—change from third to fourth application of force; P4E—change from fourth application of force back to baseline.

On average, pDVOS measured a 150% change in HbT in response to 1 N/mm² of pressure. An increase in signal at each change in applied force was observed for both the 780 nm and 850 nm wavelengths. Upon release of force, the pDVOS signal for both wavelengths (FIG. 7A) and the resultant HbT (FIG. 7B) returned to baseline.

A Spearman's rank-order correlation analysis determined that there was a strong correlation (R=−0.77, p<0.05) between location and the percent change in HbT at P34 (see FIG. 4B). Spearman's rank-order analysis also found a strong correlation (R=−0.7, p<0.05) between the percent change in HbT at the P12 and P4E interval changes (see FIG. 4B).

A change in signal relative to applied force was observed for all 3 of the healthy volunteers that data was acquired from. On average, HbT changed by 150% for every 1 N/mm² change in pressure (FIG. 4A). These results confirm that one can simultaneously collect pressure and optical data in the small vessels of the lower extremities using the disclosed pDVOS probe. Additionally, the results indicate that there is a hemodynamic response to pressure at multiple vascular locations in the lower extremities.

There was a strong correlation (R=−0.77) between the vascular site and the percent change in HbT when switching from the third to fourth interval (P34) of applied force regardless irrespective of participant. This suggests that the relationship between pressure and hemodynamics may be artery specific and serve as a useful tool in monitoring vasculature.

The strong correlation between the P12 and P4E interval changes, irrespective of location and participant, was unexpected. It was expected that the P01 and P4E interval changes to be more closely related, as they monitor the first application of force to the tissue of interest and the final release of applied force on that same tissue. The observed behavior may have been caused by poor adhesion between the pDVOS probe and the tissue, which could have led to the device slipping on the skin at the first addition of force. Poor adhesion could make the percent change in HbT measured between the P01 interval change unreliable.

Given the difficulty in monitoring small blood vessels in the feet with existing technology, the pDVOS probe may be a good option to assess occlusions in the arteries of the feet. The combination of optical data and applied pressure may provide valuable information on vascular health that could lead to better diagnostics and treatment for PAD patients. It is expected that the change in HbT between the intervals of applied force to be significantly smaller in PAD patients as compared to the data observed in this preliminary study.

These preliminary results confirm that one can simultaneously collect pressure and optical data in different locations on the lower extremities using the pDVOS probe. The strong correlation between P34 and vascular site indicates that the relationship between pressure and hemodynamics may be useful in monitoring vasculature. Given the difficulty in monitoring small blood vessels in the feet, the pDVOS probe may be a good way to assess occlusions in these arteries.

The following publications are each hereby included by reference in their entirety:
H. Johnston-Cox, D. Kadian-Dodov, and J. W. Olin, “Diagnosis and management of diseases of the peripheral arteries” in Fuster and Hurst's The Heart, 15th ed., Chapter 26, V. Fuster, et al. Eds. McGraw Hill (2022).
A. Stoyioglou and M. R. Jaff, “Medical treatment of peripheral arterial disease: a comprehensive review,” J. Vasc. Interv. Radiol. 15(11), 1197-1207 (2004).
H. L. Gornik and J. A. Beckman, “Cardiology patient page. Peripheral arterial disease,” Circulation 111(13), e169-e172 (2005).
R. A. G. Patel, R. Sakhuja, C. J. White, “The Medical and Endovascular Treatment of PAD: A Review of the Guidelines and Pivotal Clinical Trials,” Curr. Probl. Cardiol. 45(7), 100402-100429 (2020).
J. D. Santilli and S. M. Santilli, “Chronic Critical Limb Ischemia: Diagnosis, Treatment and Prognosis,” Am. Fam. Physician. 59(7), 1899-1908 (1999).
J. A. Beckman, P. A. Schneider, and M. S. Conte. “Advances in Revascularization for Peripheral Artery Disease: Revascularization in PAD. Circulation Research,” 128(12), 318261 (2021). [doi:10.1161/CIRCRESAHA.121.318261]
J. H. Rogers and J. R. Laird. “Overview of New Technologies for Lower Extremity Revascularization,” Circulation. 116(18), 715433 (2007). [doi: 10.1161/CIRCULATIONAHA.107.715433]
B. H. Gray, et al. “High incidence of restenosis/reocclusion of stents in the percutaneous treatment of long-segment superficial femoral artery disease after suboptimal angioplasty,” J Vasc Surgery. 25(1), 74-83 (1997). [doi:10.1016/S0741-5214 (97) 70323-9]
T. Yoshimura, E. Suzuki, K. Egawa, Y. Nishio, H. Maegawa, S. Morikawa, T. Inubushi, A. Hisatomi, K. Fujimoto, and A. Kashiwagi, “Low blood flow estimates in lower-leg arteries predict cardiovascular events in Japanese patients with type 2 diabetes with normal ankle-brachial indexes,” Diabetes Care 29(8), 1884-1890 (2006).
D. Xu, J. Li, Li. Zou, Y. Xu, D. Hu, S. L. Pagoto, and Y. Ma, “Sensitivity and specificity of the ankle—brachial index to diagnose peripheral artery disease: a structured review,” Vasc. Med. 15(5), 361-369 (2010).
A. Marone, J. W. Hoi, C. J. Fong, Y. Kim, H. K. Kim, D. R. Bajakian, and A. H. Hielscher, “Using dynamic vascular optical spectroscopy to evaluate peripheral arterial disease (PAD) in patients who undergo a vascular intervention,” in Optical Tomography and Spectroscopy of Tissue XIII, (SPIE 2019), pp. 108740E.
A. Marone, J. W. Hoi, M. Khalil, H. K. Kim, G. Shrikhande, R. Dayal, D. Bajakian, and A. H. Hielscher, “Modeling of the hemodynamics in the feet of patients with peripheral artery disease”. Biomedical Optics Express. 10(657), (2019).
N. Maheshwari, A. Marone, M. Altoć, H. K. Kim, D. R. Bajakian, A. H. Hielscher, “Pilot study on monitoring ulcer healing with diffuse optical imaging in a patient cohort affected by peripheral arterial disease (PAD),” in Optical Diagnostics and Sensing XXII: Toward Point-of-Care Diagnostics, (SPIE 2022), pp. 119680A.
U.S. Patent Application Publication No. 2022/0369941 A1 published on Nov. 24, 2022
U.S. Pat. No. 11,439,312 issued on Sep. 13, 2022
A. Marone, J. W. Hoi, C. J. Fong, Y. Kim, H. K. Kim, D. R. Bajakian, and A. H. Hielscher, “Using dynamic vascular optical spectroscopy to evaluate peripheral arterial disease (PAD) in patients who undergo a vascular intervention,” in Optical Tomography and Spectroscopy of Tissue XIII, (SPIE 2019), pp. 108740E.
A. Marone, J. W. Hoi, M. Khalil, H. K. Kim, G. Shrikhande, R. Dayal, D. Bajakian, and A. H. Hielscher, “Modeling of the hemodynamics in the feet of patients with peripheral artery disease”. Biomedical Optics Express. 10(657), (2019).
N. Maheshwari, A. Marone, M. Altoé, H. K. Kim, D. R. Bajakian, A. H. Hielscher, “Pilot study on monitoring ulcer healing with diffuse optical imaging in a patient cohort affected by peripheral arterial disease (PAD),” in Optical Diagnostics and Sensing XXII: Toward Point-of-Care Diagnostics, (SPIE 2022), pp. 119680A.
Maheshwari, N., Marone, A., Altoé, M., Kim, S. H., Bajakian, D. R., & Hielscher, A. H. (2022). Postintervention monitoring of peripheral arterial disease wound healing using dynamic vascular optical spectroscopy. Journal of biomedical optics, 27(12), 125002.
Marone, A., Maheshwari, N., Kim, S. H., Bajakian, D. R., & Hielscher, A. (2022). Dynamic vascular optical spectroscopy for monitoring peripheral arterial disease patients undergoing a surgical intervention. Frontiers in Photonics.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A medical pressure probe device comprising:
a body having a proximal end and a distal end, with a distal face;
at least one optical sensor and at least one light source positioned on the distal face of the body, and at least one force sensor positioned inside the body; and
a handle interfaced with the body and movable relative to the body along a path between a neutral position and an engaged position, the handle comprising a spring mechanism that contacts the at least one force sensor when the handle is in the engaged position to measure the applied pressure on the body from the handle, wherein the spring mechanism biases the handle to the neutral position.

2. The device of claim 1, wherein the spring mechanism comprises first and second portions connected by a spring, wherein the second portion contacts the at least one force sensor.

3. The device of claim 2, further comprising a guide positioned in the body configure to align the spring mechanism with the at least one force sensor.

4. The device of claim 3, wherein the at least one optical sensor comprises first, second and third optical sensors, and the at least one light source comprises between 1 and 9 light sources.

5. The device of claim 4, wherein the at least one light source comprises first, second and third light sources, and wherein the first light source is set to a wavelength of 660 nm, the second light source is set to a wavelength of 880 nm, and the third light source is set to a wavelength between 520 nm and 535 nm.

6. The device of claim 5, wherein the at least one optical sensor and at least one light source are arranged in one or more groupings on the distal face of the body, and wherein the one or more groupings are arranged in a pattern on the distal face of the body.

7. The device of claim 6, wherein a portion of the handle fits over the proximal end of the body.

8. The device of claim 7, wherein the body and handle are slidably attached or engaged.

9. The device of claim 8, wherein the spring mechanism and the guide comprise similar cross-sectional size and shape to center the spring mechanism in the guide.

10. The device of claim 9, wherein at least one of the body and the handle comprise retaining means for limiting the travel of the handle relative to the body.

11. The device of claim 10, wherein the retaining means comprise end caps, openings, posts, slots, grooves, tabs, alignment features, and combinations thereof.

12. The device of claim 11, further comprising a distal peripheral rim at least partially surrounding the distal face of the body.

13. The device of claim 12, further comprising an adhesive at least partially covering at least one of the distal face and distal peripheral rim.

14. The device of claim 1, further comprising:
a computing system communicatively connected to the sensors and at least one light source, comprising a processor and a non-transitory computer-readable medium with instructions stored thereon, which when executed by a processor, perform steps comprising:
supplying light via the at least one light source to a target site of a subject;
capturing measurements from the at least one optical sensor and the at least one force sensor;
calculating oxygenated, deoxygenated, and total hemoglobin concentrations, and oxygen saturation, based on the captured measurements;
calculating a pulse amplitude based on the captured measurements;
displaying the pulse amplitude;
displaying the applied pressure; and
recalculating pulse amplitude and hemoglobin concentrations based on the applied pressure.

15. The device of claim 14, further comprising the step of:
displaying the applied pressure with a threshold range or scale indicating an optimal applied pressure range.

16. The device of claim 14, further comprising the step of:
providing feedback to a user if the applied pressure is inside or outside an optimal applied pressure range.

17. The device of claim 16, wherein the feedback is provided with an interface for the device, wherein the interface comprises at least one of a speaker, a display, and a graphical user interface (GUI).

18. A pulse amplitude measurement method, comprising:
providing the device of claim 1;
positioning the distal face of the body at a target site on a subject;
at least partially adhering the distal face to the target site such that the at least one optical sensor and at least one light source are contacting the target site;
interfacing the handle with the body of the device;
supplying light via the at least one light source;
pushing on the handle to the engaged position to apply pressure to the target site;
capturing measurements comprising light intensity response and applied pressure;
calculating the oxygenated, deoxygenated, and total hemoglobin concentrations. and oxygen saturation. based on the captured measurements;
calculating a pulse amplitude based on the captured measurements; and
displaying the pulse amplitude.

19. The method of claim 18, further comprising:
displaying the applied pressure;
recalculating the pulse amplitude and total hemoglobin concentrations based on the applied pressure.

20. The method of claim 19, further comprising:
calculating a heart rate; and
displaying the heart rate.

21. The method of claim 18, further comprising the step of:
displaying the applied pressure with a threshold range or scale indicating an optimal applied pressure range.

22. The method of claim 21, further comprising the step of:
providing feedback to a user of the device if the applied pressure is inside or outside an optimal pressure range.

23. The device of claim 22, wherein the feedback is provided with an interface for the device, wherein the interface comprises at least one of a speaker, a display, and a graphical user interface (GUI).
